SEQUENCE LISTING (59 sequences)

SEQ ID NO: 1 - DNA, 82 bp, Artificial Sequence
  synthetic sequence
gttttagagctagaaatagcaagttaaaataaggctagtccgttatcaacttgaaaaagtggcaccgagtcggtgctttttt

SEQ ID NO: 2 - DNA, 20 bp, Artificial Sequence
  synthetic sequence
gaccctggaaaagctgatga

SEQ ID NO: 3 - DNA, 20 bp, Artificial Sequence
  synthetic sequence
gaagttcgagggcgacaccc

SEQ ID NO: 4 - DNA, 20 bp, Homo sapiens
  misc_feature (1)..(1): n=G or A
naccctggaaaagctgatga

SEQ ID NO: 5 - DNA, 20 bp, Homo sapiens
gtggatgacataatgctttt

SEQ ID NO: 6 - DNA, 20 bp, Homo sapiens
  misc_feature (4)..(4): n=G or A
gganccgctgcaccgaccgt

SEQ ID NO: 7 - DNA, 20 bp, Homo sapiens
  misc_feature (20)..(20): n=G or A
tgggacgcaaggcgccgtgn

SEQ ID NO: 8 - DNA, 4140 bp, Artificial Sequence
  synthetic sequence
atggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtga

SEQ ID NO: 9 - DNA, 972 bp, Artificial Sequence
  synthetic sequence
ctgcccggcgcctacaacgtcaacatcaagttggacatcacctcccacaacgaggactacaccatcgtggaacagtacgaacgcgccgagggccgccactccaccggcggcatggacgagctgtacaagggtaccagcgcgccgcgtcgagagatgggggaggctaacagaagtgtacgcgttggctgctgtcaggggtaccatggacgcgtagacaaggtgaggaacctagctcagtcatgcaccgggctgcagatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtga

SEQ ID NO: 10 - DNA, 20 bp, Artificial Sequence
  synthetic
gctgaagggcatcgacttca

SEQ ID NO: 11 - DNA, 20 bp, Artificial Sequence
  synthetic
caactacaagacccgcgccg

SEQ ID NO: 12 - DNA, 10867 bp, Artificial Sequence
  synthetic
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgctagtggatcccccgggctgcaggaattatcacaagtttgtacaaaaaagcaggcttcatcgatcaattgggatccaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctgaagttcgagggcgacacccgttttagagctagaaatagcaagttaaaataaggctagtccgttatcaacttgaaaaagtggcaccgagtcggtgcttttttagatctggatccaagcttccatggaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaaacaccgctgaagggcatcgacttcagttttagagctagaaatagcaagttaaaataaggctagtccgttatcaacttgaaaaagtggcaccgagtcggtgcttttttcatatgccatgggcggccgctctagactgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctccaactacaagacccgcgccggttttagagctagaaatagcaagttaaaataaggctagtccgttatcaacttgaaaaagtggcaccgagtcggtgcttttttggtacctctagactcgagactagtaggcctgacccagctttcttgtacaaagtggtgataattcgatatcaagcttatcgataccgtcgacctcgacgatggtcgagtaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctctaggatcccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagcggccgcgtcgagagatgggggaggctaactgagctagcttagtcatgcaccgggctgcaggaattcgcggccgcactcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgagctcggtacctttaaaaccaatgacttacaaggcagctgtaaatcttagccactttttaaaagaaaaggggggactggaagggctaattcactcccaacgaaaacaaaatctgctttttgcttgtactgggtctctctggttagaccaaatctgagcctgggagctctctggctaactagggaacccactgcttaagcctcaataaagcttgccttgagtgcttcaagtagtgtgtgcccgtctgttgtgtgactctggtaactagagatccctcaaacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagagcttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtgtggatcaactggataactcaagctaaccaaaatcatcccaaacttcccaccccataccctattaccactgccaaattacctgtggtttcatttactctaaacctgtgattcctctgaattattttcattttaaagaaattgtatttgttaaatatgtactacaaacttagtagt

SEQ ID NO: 13 - DNA, 20 bp, Artificial Sequence
  synthetic
ggtaatttgtgtttgtgtnc

SEQ ID NO: 14 - DNA, 20 bp, Artificial Sequence
  synthetic
gacagcagagaaacagcngt

SEQ ID NO: 15 - DNA, 20 bp, Artificial Sequence
  synthetic
gagaaacagcngttagttcc

SEQ ID NO: 16 - DNA, 20 bp, Artificial Sequence
  synthetic
tggctaaagtaggctttant

SEQ ID NO: 17 - DNA, 20 bp, Artificial Sequence
  synthetic
cagggctgtccgggtgagna

SEQ ID NO: 18 - DNA, 20 bp, Artificial Sequence
  synthetic
tgtccgggtgagnatggctc

SEQ ID NO: 19 - DNA, 20 bp, Artificial Sequence
  synthetic
aggcccatgcggaaaggatn

SEQ ID NO: 20 - DNA, 20 bp, Artificial Sequence
  synthetic
ggcggggnatcctttccgca

SEQ ID NO: 21 - DNA, 20 bp, Artificial Sequence
  synthetic
ggggctcaacggagagngga

SEQ ID NO: 22 - DNA, 20 bp, Artificial Sequence
  synthetic
ggcagaacctgcgggggcng

SEQ ID NO: 23 - DNA, 20 bp, Artificial Sequence
  synthetic
gaacctgcgggggcnggggc

SEQ ID NO: 24 - DNA, 20 bp, Artificial Sequence
  synthetic
attttaccagtattccngtc

SEQ ID NO: 25 - DNA, 20 bp, Artificial Sequence
  synthetic
ctgtgtgtcgagtgtacagt

SEQ ID NO: 26 - DNA, 20 bp, Artificial Sequence
  synthetic
ataaaaataagttaacactn

SEQ ID NO: 27 - DNA, 20 bp, Artificial Sequence
  synthetic
gcaggagaatggcntgaact

SEQ ID NO: 28 - DNA, 20 bp, Artificial Sequence
  synthetic
ataatatagtanggccactc

SEQ ID NO: 29 - DNA, 20 bp, Artificial Sequence
  synthetic
cataaaactgaacataccnt

SEQ ID NO: 30 - DNA, 20 bp, Artificial Sequence
  synthetic
agcaattgggcaacntcgtg

SEQ ID NO: 31 - DNA, 20 bp, Artificial Sequence
  syntetic
agacggggcctcacgangtt

SEQ ID NO: 32 - DNA, 20 bp, Artificial Sequence
  synthetic
agaaggtgctgctagttcan

SEQ ID NO: 33 - DNA, 20 bp, Artificial Sequence
  synthetic
tgtgtgtgtgtntntatatt

SEQ ID NO: 34 - DNA, 20 bp, Artificial Sequence
  synthetic
gttgtggttgccaagtaang

SEQ ID NO: 35 - DNA, 20 bp, Artificial Sequence
  synthetic
ggagtggcggggcngggggg

SEQ ID NO: 36 - DNA, 20 bp, Artificial Sequence
  synthetic
cgggacgggtccaagatgga

SEQ ID NO: 37 - DNA, 20 bp, Artificial Sequence
  synthetic
gggctgtcaatcatgctggc

SEQ ID NO: 38 - DNA, 20 bp, Artificial Sequence
  synthetic
cttttccagggtcgccatgg

SEQ ID NO: 39 - DNA, 20 bp, Artificial Sequence
  synthetic
gacaatatgtgaaaacatag

SEQ ID NO: 40 - DNA, 20 bp, Artificial Sequence
  synthetic
aatggagtacttcttgtcca

SEQ ID NO: 41 - DNA, 4221 bp, Artificial Sequence
  synthetic
atgccaactttgtacaaaaaagcaggctccaccatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggtaccggtaagcctatccctaaccctctcctcggtctcgattctacgtga

SEQ ID NO: 42 - DNA, 4885 bp, Homo sapiens
cttactccctgctgggcaggctggacagctgccctctccgtttgcccctctaccacccaaaaggcaggaggctctggagaccaggaccctgcccgccacggcctgtgtcccaggcgtgagggggtgccccacagacctctgctgagctgctgctgaatgacgccccttgggggtcctgccggaaggtcagagcaggggtgcactcccataaagaaacgcccccaggtcgggactcattcctgtgggcggcatcttgtggccatagctgcttctcgctgcactaatcacagtgcctctgtgggcagcaggcgctgaccacccaggcctgccccagaccctctcctcccttccggggcgctgcgctgggaccgatggggggcgccaggcctgtggacaccgccctgcaggggcctctccagctcactgggggtggggtgggggtcacacttggggtcctcaggtcgtgccgaccacgcgcattctctgcgctctgcgcaggagctcgcccaccctctccccgtgcagagagccccgcagctggctccccgcagggctgtccgggtgagtatggctctggccacgggccagtgtggcgggagggcaaaccccaaggccacctcggctcagagtccacggccggctgtcgccccgctccaggcgtcggcgggggatcctttccgcatgggcctgcgcccgcgctcggcgccccctccacggccccgccccgtccatggccccgtccttcatgggcgagcccctccatggccctgcccctccgcgccccacccctccctcgccccacctctcaccttcctgccccgcccccagcctccccacccctcaccggccagtcccctcccctatcccgctccgcccctcagccgccccgcccctcagccggcctgcctaatgtccccgtccccagcatcgccccgccccgcccccgtctcgccccgcccctcaggcggcctccctgctgtgccccgccccggcctcgccacgcccctacctcaccacgccccccgcatcgccacgccccccgcatcgccacgcctcccttaccatgcagtcccgccccgtcccttcctcgtcccgcctcgccgcgacacttcacacacagcttcgcctcaccccattacagtctcaccacgccccgtcccctctccgttgagccccgcgccttcgcccgggtggggcgctgcgctgtcagcggccttgctgtgtgaggcagaacctgcgggggcaggggcgggctggttccctggccagccattggcagagtccgcaggctagggctgtcaatcatgctggccggcgtggccccgcctccgccggcgcggccccgcctccgccggcgcagcgtctgggacgcaaggcgccgtgggggctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcaacagcagcaacagcagcaacagcagcaacagcagcaacagcagcaacagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccgtgagtttgggcccgctgcagctccctgtcccggcgggtcccaggctacggcggggatggcggtaaccctgcagcctgcgggccggcgacacgaacccccggccccgcagagacagagtgacccagcaacccagagcccatgagggacacccgccccctcctggggcgaggccttcccccacttcagccccgctccctcacttgggtcttcccttgtcctctcgcgaggggaggcagagccttgttggggcctgtcctgaattcaccgaggggagtcacggcctcagccctctcgcccttcgcaggatgcgaagagttggggcgagaacttgtttctttttatttgcgagaaaccagggcgggggttcttttaactgcgttgtgaagagaacttggaggagccgagatttgctcagtgccacttccctcttctagtctgagagggaagagggctgggggcgcgggacacttcgagaggaggcggggtttggagctggagagatgtgggggcagtggatgacataatgcttttaggacgcctcggcgggagtggcggggcagggggggggcggggagtgagggcgcgtccaatgggagatttcttttcctagtggcacttaaaacagcctgagatttgaggctcttcctacattgtcaggacatttcatttagttcatgatcacggtggtagtaacacgattttaagcaccacctaagagatctgctcatctaagcctaagttggtctgcaggcgtttgaatgagttgtggttgccaagtaaagtggtgaacttacgtggtgattaatgaaattatcttaaatattaggaagagttgattgaagttttttgcctatgtgtgttgggaataaaaccaacacgttgctgatggggaggttaattgccgagggatgaatgaggtgtacattttaccagtattccagtcaggcttgccagaatacggggggtccgcagactccgtgggcatctcagatgtgccagtgaaagggtttctgtttgcttcattgctgacagcttgttactttttggaagctaggggtttctgttgcttgttcttggggagaatttttgaaacaggaaaagagagaccattaaaacatctagcggaaccccaggactttccctggaagtctgtgtgtcgagtgtacagtaggagttaggaagtactctggtgcagttcaggcctttctcttacctctcagtattctatttccgatctggatgtgtcccagatggcatttggtaagaatatctctgttaagactgattaatttttagtaatatttcttgttctttgtttctgttatgatccttgtctcgtcttcaaagtttaattagaaaatgattcggagagcagtgttagcttatttgttggaataaaatttaggaataaattattctaaaggatggaaaaactttttggatatttggagaaattttaaaacaatttggcttatctcttcagtaagtaatttctcatccagaaatttactgtagtgcttttctaggaggtaggtgtcataaaagttcacacattgcatgtatcttgtgtaaacactaaacagggctcctgatgggaaggaagacctttctgctgggctgcttcagacacttgatcattctaaaaatatgccttctctttcttatgctgatttgacagaacctgcatttgcttatcttcaaaatatgggtatcaagaaatttcctttgctgccttgacaaaggagatagattttgtttcattactttaaggtaatatatgattaccttatttaaaaaatttaatcaggactggcaaggtggcttacacctttaatccgagcactttgggaggcctaggtggacgaatcacctgaggtcaggagtttgagaccagcctggctaacatggtgaaaccctgtctctactaaaaatacaaaaattagctggtcatggtggcacgtgcctgtaatccaagctacctgggaggctgaggcaggaaaatcgcttgaacccgggaggcagagtctgcagtgagttgagatcacgccactgcactccagcctgggtgacagagcgagactctatctcaaaaaaaattttttttaatgtattatttttgcataagtaatacattgacatgatacaaattctgtaattacaaaagggcaataattaaaatatcttccttccacccctttcctctgagtacctaactttgtccccaagaacaagcactatttcagttcctcatgtatcctgccagatataacctgttcatattgtaagatagatttaaaatgctctaaaaacaaaagtagtttagaataatatatatctatatattttttgagatgtagtctcacattgtcacccaggctggagtgcagtgatacaatctcggctcactgcagtctctgcctcccaggttcaaatgcttctcctgcctcagccttctgagtagctgggattacaggcgcccaccaccatgtccagctaatttttgtatttttagtagagatggggtttcaccatgttggccaggctggtcttgaactcctgaccttgtgatctgtccacctcggcctcccaaagtgctgggattacaggtgtgagccaccatgcctggctagaataataacttttaaaggttcttagcatgctctgaaatcaactgcattaggtttatttatagttttatagttattttaaataaaatgcatatttgtcatatttctctgtattttgctgttgagaaaggaggtattcactaattttgagtaacaaacactgctcacaaagtttggattttggcagttctgttcacgtgcttcagccaaaaaatcctcttctcaaagtaagattgatgaaagcaatttagaaagtatctgttctgtttttatggctcttgctctttggtgtggaactgtggtgtcacgccatgcatgggcctcagtttatgagtgtttgtgctctgctcagcatacaggatgcaggagttccttatggggctggctgcaggctcagcaaatctagcatgcttgggagggtcctcacagtaattaggaggcaattaatacttgcttctggcag

SEQ ID NO: 43 - DNA, 4840 bp, Homo sapiens
cttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcaacagcagcaacagcagcaacagcagcaacagcagcaacagcagcaacagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccgtgagtttgggcccgctgcagctccctgtcccggcgggtcccaggctacggcggggatggcggtaaccctgcagcctgcgggccggcgacacgaacccccggccccgcagagacagagtgacccagcaacccagagcccatgagggacacccgccccctcctggggcgaggccttcccccacttcagccccgctccctcacttgggtcttcccttgtcctctcgcgaggggaggcagagccttgttggggcctgtcctgaattcaccgaggggagtcacggcctcagccctctcgcccttcgcaggatgcgaagagttggggcgagaacttgtttctttttatttgcgagaaaccagggcgggggttcttttaactgcgttgtgaagagaacttggaggagccgagatttgctcagtgccacttccctcttctagtctgagagggaagagggctgggggcgcgggacacttcgagaggaggcggggtttggagctggagagatgtgggggcagtggatgacataatgcttttaggacgcctcggcgggagtggcggggcagggggggggcggggagtgagggcgcgtccaatgggagatttcttttcctagtggcacttaaaacagcctgagatttgaggctcttcctacattgtcaggacatttcatttagttcatgatcacggtggtagtaacacgattttaagcaccacctaagagatctgctcatctaagcctaagttggtctgcaggcgtttgaatgagttgtggttgccaagtaaagtggtgaacttacgtggtgattaatgaaattatcttaaatattaggaagagttgattgaagttttttgcctatgtgtgttgggaataaaaccaacacgttgctgatggggaggttaattgccgagggatgaatgaggtgtacattttaccagtattccagtcaggcttgccagaatacggggggtccgcagactccgtgggcatctcagatgtgccagtgaaagggtttctgtttgcttcattgctgacagcttgttactttttggaagctaggggtttctgttgcttgttcttggggagaatttttgaaacaggaaaagagagaccattaaaacatctagcggaaccccaggactttccctggaagtctgtgtgtcgagtgtacagtaggagttaggaagtactctggtgcagttcaggcctttctcttacctctcagtattctatttccgatctggatgtgtcccagatggcatttggtaagaatatctctgttaagactgattaatttttagtaatatttcttgttctttgtttctgttatgatccttgtctcgtcttcaaagtttaattagaaaatgattcggagagcagtgttagcttatttgttggaataaaatttaggaataaattattctaaaggatggaaaaactttttggatatttggagaaattttaaaacaatttggcttatctcttcagtaagtaatttctcatccagaaatttactgtagtgcttttctaggaggtaggtgtcataaaagttcacacattgcatgtatcttgtgtaaacactaaacagggctcctgatgggaaggaagacctttctgctgggctgcttcagacacttgatcattctaaaaatatgccttctctttcttatgctgatttgacagaacctgcatttgcttatcttcaaaatatgggtatcaagaaatttcctttgctgccttgacaaaggagatagattttgtttcattactttaaggtaatatatgattaccttatttaaaaaatttaatcaggactggcaaggtggcttacacctttaatccgagcactttgggaggcctaggtggacgaatcacctgaggtcaggagtttgagaccagcctggctaacatggtgaaaccctgtctctactaaaaatacaaaaattagctggtcatggtggcacgtgcctgtaatccaagctacctgggaggctgaggcaggaaaatcgcttgaacccgggaggcagagtctgcagtgagttgagatcacgccactgcactccagcctgggtgacagagcgagactctatctcaaaaaaaattttttttaatgtattatttttgcataagtaatacattgacatgatacaaattctgtaattacaaaagggcaataattaaaatatcttccttccacccctttcctctgagtacctaactttgtccccaagaacaagcactatttcagttcctcatgtatcctgccagatataacctgttcatattgtaagatagatttaaaatgctctaaaaacaaaagtagtttagaataatatatatctatatattttttgagatgtagtctcacattgtcacccaggctggagtgcagtgatacaatctcggctcactgcagtctctgcctcccaggttcaaatgcttctcctgcctcagccttctgagtagctgggattacaggcgcccaccaccatgtccagctaatttttgtatttttagtagagatggggtttcaccatgttggccaggctggtcttgaactcctgaccttgtgatctgtccacctcggcctcccaaagtgctgggattacaggtgtgagccaccatgcctggctagaataataacttttaaaggttcttagcatgctctgaaatcaactgcattaggtttatttatagttttatagttattttaaataaaatgcatatttgtcatatttctctgtattttgctgttgagaaaggaggtattcactaattttgagtaacaaacactgctcacaaagtttggattttggcagttctgttcacgtgcttcagccaaaaaatcctcttctcaaagtaagattgatgaaagcaatttagaaagtatctgttctgtttttatggctcttgctctttggtgtggaactgtggtgtcacgccatgcatgggcctcagtttatgagtgtttgtgctctgctcagcatacaggatgcaggagttccttatggggctggctgcaggctcagcaaatctagcatgcttgggagggtcctcacagtaattaggaggcaattaatacttgcttctggcagtttcttattctccttcagattcctatctggtgtttccctgactttattcattcatcagtaaatatttactaaacatgtactatgtgcctggcactgttataggtgcagggctcagcagtgagcagacaaagctctgccctcgtgaagctttcattctaatgaaggacatagacagtaagcaagatagataagtaaaatatacagtacgttaatacgtggaggaacttcaaagcagggaaggggatagggaaatgtcagggttaatcgagtgttaacttatttttatttttaaaaaaattgttaagggctttccagcaaaacccagaaagcctgctagacaaattccaaaagagctgtagcactaagtgttgacatttttattttattttgttttgttttgttttttttgagacagttcttgctctatcagccaggctggagtgcactagtgtgatcttggctcactgcaacctctgcctcttgggttcaagtgattctcatgcctcagcctcctgtttagctgggattatagacatgcactgccatgcctgggtaattttttttttttcccccgagacggagtcttgctctgtcgcccaggctggagtgcagtggcgcgatctcagctcactgcaagctccgcttcccgagttcacgccattctcctgcctcagtctcccaagtagctgggactacaggcgcctgccaccacgtccagctaatttttttgtatttttaatagagacggggtttcaccgtgttagccaggatgatcttgatctcctgacctcgtcatccgccgaccttgtgatccgcccacctcggcctcccaaagtgctgggattacaggcatgagccactgtgcccggccacgcctgggtaatttttgtatttttagtagagatggggttttgccatgatgagcaggctggtctcgaactcccggcctcatgtgatctgcctgccttggcctcccaaagtgctaggattacaggcatgagccaccatacctggccagtgttgatattttaaatacggtgttcagggaaggtccactgagaagacagctttttttttttttttttttggggttggggggcaaggtcttgctctttaacccaggctggaatgcagtatcactatcgtagctcacttcagccttgaactcctgggctcaagtgatcctcccacctcaacctcacaatgtgttgggactataggtgtgagccatcacacctggccagatgatggcttttgagtaaagacctcaagcgagttaagagtctagtgtaagggtgtatgaagtagtggtattccagatggggggaacaggtccaaaatcttcctgtttcaggaatagcaaggatgtcattttagttgggt

SEQ ID NO: 44 - DNA, 21 bp, Artificial Sequence
  synthetic
tgtgtgcccgtctgttgttgt

SEQ ID NO: 45 - DNA, 20 bp, Artificial Sequence
  synthetic
gagtcctgcgtcgagagagc

SEQ ID NO: 46 - DNA, 21 bp, Artificial Sequence
  synthetic
atggacggccgctcaggttct

SEQ ID NO: 47 - DNA, 21 bp, Artificial Sequence
  synthetic
gctcagcaccggggcaatgaa

SEQ ID NO: 48 - DNA, 30 bp, Artificial Sequence
  synthetic
acggcgagttcatctacaaggtgaagctgc

SEQ ID NO: 49 - DNA, 20 bp, Artificial Sequence
  synthetic
tgacaaatggaagtagcacg

SEQ ID NO: 50 - DNA, 30 bp, Artificial Sequence
  synthetic
ttgtactccagcttgtgccccaggatgttg

SEQ ID NO: 51 - DNA, 23 bp, Artificial Sequence
  synthetic
gagatgagtttttgttcgaaggg

SEQ ID NO: 52 - DNA, 25 bp, Artificial Sequence
  synthetic
ttgctgtgtgaggcagaacctgcgg

SEQ ID NO: 53 - DNA, 20 bp, Artificial Sequence
  synthetic
tgcagcggctcctcagccac

SEQ ID NO: 54 - DNA, 20 bp, Artificial Sequence
  synthetic
cacttcacacacagcttcgc

SEQ ID NO: 55 - DNA, 20 bp, Artificial Sequence
  synthetic
tgctgctggaaggacttgag

SEQ ID NO: 56 - DNA, 20 bp, Artificial Sequence
  synthetic
tgacaaatggaagtagcacg

SEQ ID NO: 57 - DNA, 23 bp, Artificial Sequence
  synthetic
gagatgagtttttgttcgaaggg

SEQ ID NO: 58 - DNA, 22 bp, Artificial Sequence
  synthetic
cctcctcacttcttttctatcg

SEQ ID NO: 59 - DNA, 21 bp, Artificial Sequence
  synthetic
agcattatgtcatccactacc

(19) United States
(12) Patent Application Publication
(10) Pub. No.: US 2017/0224843 A1
(43) Pub. Date: Aug. 10, 2017

(54) GENOME EDITING FOR THE TREATMENT OF HUNTINGTON'S DISEASE

(71) Applicant: CENTRE HOSPITALIER UNIVERSITAIRE VAUDOIS (CHUV), Lausanne (CH)

(72) Inventors: Nicole DEGLON, Curtilles (CH); Nicolas MERIENNE, Montpreveyres (CH)

(73) Assignee: CENTRE HOSPITALIER UNIVERSITAIRE VAUDOIS (CHUV), Lausanne (CH)

(21) Appl. No.: 15/501,965

(22) PCT Filed: Aug. 4, 2015
(86) PCT No.: PCT/EP2015/067986

§ 371 Date: Feb. 6, 2017

(30) Foreign Application Priority Data
Aug. 4, 2014 (EP) 14179767.0

(51) Int. Cl. A61K 48/00; C12N 15/11; A61K 38/46; A61K 38/17; C12N 15/86

(52) U.S. Cl. CPC A61K 48/005; A61K 38/1709; C12N 15/86; A61K 38/465; C12N 15/11; C12N 2750/14143; C12N 2310/20; C12N 2310/3513

(57) ABSTRACT

A treatment of Huntington's disease (HD) using the Clustered-Regularly Interspaced Short Palindromic Repeats (CRISPR) system. This technology offers the possibility to design a small RNA (sgRNA), which is incorporated into a CRISPR-associated protein (Cas9) to recognize and induce DNA double-strand breaks at a specific target location. In the context of HD, this allows to block the expression of the mutant huntingtin or repair the CAG expansion causing the disease.

### FIELD OF THE INVENTION

The invention relates to the treatment of Huntington's disease (HD) using the Clustered-Regularly Interspaced Short Palindromic Repeats (CRISPR) system. This technology offers the possibility to design a single guide RNA (sgRNA), which is incorporated into a CRISPR-associated protein (Cas9) to recognize and induce DNA double-strand breaks at a specific target location. DNA double-strand breaks will be repaired by cellular machinery either by non-homologous end joining (NHEJ) or homologous recombination (HR) in the presence of a donor sequence for HD gene repair. In the context of HD, this allows to block the expression of the mutant huntingtin (mHTT) or repair the CAG expansion causing the disease.

### BACKGROUND OF THE INVENTION

Huntington's disease (HD) is a monogenic neurodegenerative disease characterized by a global impairment leading to death in 15-20 years. Although precise mechanisms leading to neuronal death are not yet understood, a pathologic expansion of CAG repeats (more than 40 CAG) at the end of the exon 1 of the huntingtin gene (HTT) has been identified as the cause of HD. The huntingtin locus located in chromosome 4 is large, spanning 180 kb and consisting of 67 exons. In HD, the huntingtin protein (HTT) forms aggregates in cells, which interfere with normal cellular functions (1). In addition, normal function of HTT is altered and new pathologic interactions of mutant HTT (mHTT) with partners also participate to the neuronal death observed in HD (2). Currently, there are no curative treatments for HD but experimental approaches based on drug, cell and gene therapy are under investigation (3). One of the most promising is the silencing of mHTT with small-hairpin RNA (shRNA), which will either degrade wild-type (WT) and mHTT mRNA (4, 5) or induce an allele-specific silencing based on the presence of single nucleotide polymorphism (SNP) in the HTT genome and selective silencing of mHTT mRNA (6). Most of the patients are heterozygous for the disease-causing mutation. SNP with a high frequency in the human population have been used to selectively reduce the expression of the mutant HTT allele containing the matching SNP while leaving the expression of the WT allele (mismatched SNP) unaltered.

Recently, approaches modulating HTT gene expression have also been described, in particular with zinc finger and TALE repressors. Allele and non-allele specific targeting of mutant HTT using ZFP- or TALE-repressors were used to block the expression of the mutant HTT (7). ZFP targeting the expanded CAG were shown to preferentially repress the expression of the human HTT containing longer CAG repeats in vitro. In the R6/2 mice, the injection of an AAV2/1 vector expressing ZF11×Hunt-Kox-1 reduces the level of mutant HTT mRNA and the accumulation of misfolded HTT in the striatum by 40% on average. Clasping behavior and motor coordination were also improved (7).

Based on the same principle, WO 2013/130824 (Sangamo Biosciences, Inc.) and Garriga-canut et al 2012 (7) disclose methods and compositions for treating or preventing Huntington's disease. In particular, these documents provide methods and compositions for modulating expression of a mutant HTT allele so as to treat Huntington's disease. Also provided are methods and compositions for generating animal models of Huntington's disease. The invention is based on ZFN and TALE with engineered DNA binding domain targeting the HTT gene. These proteins are in operative linkage with regulatory (or functional domain) as part of a fusion protein. The functional domain can be a transcriptional activation domain or a transcriptional repression domain. These strategies will therefore either repress or activate the expression of the human HTT gene (global or allele-specific). However, such methods require a permanent expression of the therapeutic gene and are not definitely modifying the HTT DNA sequence.

WO 2014/093701 (Feng Zhang, MIT, USA) and Cong et al 2014 (8) relate to compositions, methods, applications and screens used in functional genomics that focus on gene function in a cell and that may use vector systems and other aspects related to Clustered Regularly Interspaced Short Palindromic Repeats (CRISPR)-Cas systems and components thereof. Provided are vectors and vector systems, some of which encode one or more components of a CRISPR complex, as well as methods for the design and use of such vectors. Also provided are methods of directing CRISPR complex formation in eukaryotic cells and methods for utilizing the CRISPR-Cas system.

The present invention is based on entirely new and innovative strategies for allele or non-allele-specific HTT editing and is also describing gene repair strategies targeting the promoter, transcription and/or translation start sites and/or SNP in the HTT gene. HTT gene disruption based on sgRNA targeting the CAG expansion or double-strand break with overhang (2-4 sgRNA and Cas nickase) are not considered in the present invention.

The recently described method based on the bacterial clustered regularly interspaced short palindromic repeats (CRISPR) system is a promising tool for gene editing in mammals and offers a unique opportunity to irreversibly modify the HTT gene itself and develop therapeutic strategies based on DNA editing/repair (9). The CRISPR system contains two components: (i) a dual crRNA:tracrRNA which was engineered as an artificial single guide RNA (sgRNA) of approximately 20 nucleotides recognizing the target sequence by Watson-Crick base-pairing fused with the tracrRNA at the 3′ side of the guide sequence that bind to Cas9 (tracrRNA), (ii) the second component of the system is the CRISPR-associated protein Cas9 nuclease cleaving at the target site. Any sequence of approximately 23 nucleotides including the protospacer adjacent motif at the 3′ end of the target sequence (PAM=NGG or NAG for Cas9) and located on both strands of DNA could be a CRISPR target sequence, which provides a large number of potential targets for each gene and greatly facilitates the development of DNA repair strategies compared to the other systems. Sternberg and collaborators demonstrated that both binding and cleavage of DNA by Cas9 require recognition of the short trinucleotide PAM (10). The DNA endonuclease binds targeted nucleotides to perform double-strand breaks (DSB) and non-homologous end joining (NHEJ) with insertions or deletions (indels) in the sequence created by the cellular repair machinery. When an exogenous DNA sequence with homology with the target sequence is added, HR and integration into the desired locus is occurring after the DSB.

One prerequisite for the development of in vivo gene repair strategy in the CNS is an efficient delivery system. The design, production, and efficiency of gene transfer vectors, has improved remarkably over time, leading to safer transduction and long-term and robust transgene expression in the brain (11). This has led to the initiation of several phase I/II clinical trials with adeno-associated vectors (AAV) and lentiviral vectors (LV) in patients suffering from Parkinson's, Alzheimer's, Batten, adrenoleukodystrophy or Canavan's disease (12-14). The identification of numerous natural or chimeric AAV serotypes with variable capsid proteins has been exploited to modulate the entry into the host and enhance transduction efficiencies in the CNS (15, 16). Most studies with LV have been performed with VSV-G (vesicular stomatitis virus glycoprotein G) pseudotyped vectors, but the tropism of the vector could be altered with the use of heterologous envelopes. Their relatively large cloning capacity (8-9 kb compared with 4-5 kb for AAV) is particularly advantageous for the cloning of complex expression cassettes with large cDNAs.

Current approaches based on HTT silencing (RNAi, ASO) or modulation of HTT expression (ZFN/TALEN-repressors) required a continuous expression of the compounds to maintain the therapeutic benefits and partial HTT silencing or repression are obtained. In contrast, there is a need to engineer an effective CRISPR/Cas9 system inducing indels or DNA repair, with the goal to provide permanent and irreversible reactions, leading to a complete HTT gene disruption or repair and ensuring a long-lasting therapeutic benefit.

### BRIEF DESCRIPTION OF THE INVENTION

One of the objects of the present invention is to provide a kit for the treatment of Huntington's disease (HD) for allele or non-allele-specific huntingtin (HTT) gene editing or repair comprising:
a gene delivery vector consisting of at least one viral vector selected among adeno-associated vector serotypes (AAV) and/or lentiviral vectors (LV);
a Cas9 being human codon-optimized or fused to an epitope tag selected among the group of FLAG, His, myc, Tap, HA, V5. Preferably Cas9 is fused to fused to an epitope tag (e.g V5 tag).
at least one artificial single guide RNA (sgRNA) having a total size from 63-115 nucleotides comprising a tracrRNA sequence of 48-85 nucleotides long (preferably 82 bp) and a crRNA sequence of 15-30 nucleotides (preferably 20 bp) recognizing the sequence of the HTT gene around the expanded CAG repeat mutation (ENSG00000197386, position of the first CAG repeat in exon 1: 3′074′877-3′074′879), and comprising the region upstream of the HTT gene (position 3066800) up to the beginning of intron 2 (position 3087600), wherein said crRNA sequence binds directly upstream of the required 5′-NGG/NAG-3′ protospacer adjacent motif (PAM) and whereas said crRNA sequence base-pairs with the target HTT sequence and Cas9 mediates a double-stranded break (DSB) 3-4 bp upstream of said PAM.

Another object of the present invention is to provide a kit for the treatment of Huntington's disease, for use in a method for non-allele-specific HTT inactivation of the human HTT wild-type (WT) and mutant alleles. For this, a region around the expanded CAG repeat mutation and comprising the region upstream of the HTT gene (position 3066800) up to the beginning of intron 2 (position 3087600) are targeted for the selection of the sgRNA.

A further object of the invention is to provide a kit for the treatment of Huntington's disease, for use in a method for non-allele-specific HTT gene repair based on HR with a DNA template containing a WT HTT sequence.

Also provided is a kit for the treatment of Huntington's disease, for use in a method for allele-specific HTT inactivation of the human mutant HTT gene wherein said at least one sgRNA is capable of recognizing SNP sequences located upstream a PAM along the HTT gene with a high frequency of heterozygosity in the human population at least >5% (according to dbSNP or 1000 Genome Project database).

The present invention also provides a kit for the treatment of Huntington's disease for use in a method for mutant HTT gene repair based on HR with a DNA template containing a wild-type HTT sequence, wherein said at least one sgRNA is capable of recognizing SNP sequences located upstream a PAM along the HTT gene with a high frequency of heterozygosity in the human population at least >5% (according to dbSNP or 1000 Genome Project database).

Other objects and advantages of the invention will become apparent to those skilled in the art from a review of the ensuing detailed description, which proceeds with reference to the following illustrative drawings, and the attendant claims.

Applicants use the CRISPR system to definitely disrupt or repair the HD mutation. Applicants target sites with the CRISPR in a region comprising the region upstream of the HTT gene (position 3066800) up to the beginning of intron 2 (position 3087600), with an emphasis around the transcription start site and the translation initiation codon ATG with the CRISPR to block the expression of the mutant HTT at the DNA level. Both Cas9 and sgRNAs are delivered in CNS structures affected in HD (neurons and/or astrocytes) of rodent expressing the human mutant HTT with virals vectors, for example VSV-G pseudotyped LV or AAV2/5 vector, expressing a human codon optimized Cas9 under the PGK, CMV or CBA promoters followed by the woodchuck post-transcriptional regulatory element (WPRE). The second one expresses one copy or multiple sgRNA under the H1 promoter or other polymerase II or III promoters and stuffer DNA to ensure independent expression of each sgRNA. Blocking the expression of mHTT in adult neurons and/or glial cells helps to rescue the HD phenotype and therefore provides therapeutic benefit.

DNA disruption/repair of Huntington's disease mutation using the CRISPR/Cas9 system represents a new and original therapeutic approach. The present invention offers the possibility to act at the DNA level with engineered nucleases to inactivate or repair a disease-causing mutation. Previous works using RNA silencing obtained a significant decrease of mHTT expression, which lead to an improved phenotype. Recently, an approach based on zinc-finger repressors was evaluated in HD mouse model brain and reached a low but significant reduction of mHTT expression. However, all these approaches need a continuous expression of the therapeutic gene. In addition, only a partial silencing of the mutant HTT is obtained and this has encouraged the development of complete/irreversible reverse genetic approaches. In the case of the genome editing strategy of the present invention, only a transient expression of the nuclease is required to induce the DSB. HTT gene disruption or repair mediated by this DSB will be permanent.

### BRIEF DESCRIPTION OF THE FIGURES

FIG. 1: Schematic representation of the CRISPR system showing the Cas9 protein, sgRNA and HTT target sequence with the adjacent PAM. The sgRNA is composed of a tracrRNA sequence of 48-85 nucleotides (scaffold sequence) and a crRNA sequence of 15-30 nucleotides base-pairing with the target sequence. The design and specific features of the resulting chimeric sgRNA are shown. Applicants analyze sequences of interest for all possible targetable sequences (preferably 20 nt) immediately 5′ to a NGG/NAG PAM motif. Unique genomic sequences were selected to minimize off-target effects (based on dedicated softwares, such as the CRISPRseek Bioconductor package to identify Target-Specific Guide RNAs for CRISPR-Cas9 Genome-Editing Systems). The sgRNA was cloned into a delivery system containing preferably, polymerase III promoters (H1, U6 and 7SK), which are leading to high expression levels.

FIG. 2: Description of the reporter gene constructs. (A) An artificial target sequence was used and cloned in viral vectors. An artificial target sequence was introduced between the mCherry-GFP sequence expressed under the PGK promoter. This target consists of 20 nucleotides carrying a STOP codon two nucleotides 5′ of the NGG PAM. Cells expressing this construct will display only mCherry fluorescence. If DSB occurs after target sequence recognition by the CRISPR system, the STOP codon will be removed and a fusion protein: mCherry-GFP will be expressed. For HR experiments, the presence of the homologous donor DNA without the STOP codon will be integrated at the desire locus. In addition, HR leads to the insertion of an NcoI restriction site at the end of the target sequence. (B) Schematic representation of the 3′ end of the human HTT (hHTT) promoter, exon 1 and 5′ end of intron 1 showing examples of sgHTT. Circles represent positions of known SNP in the hHTT gene with their corresponding name (starting with rs; dbSNP http://www.ncbi.nlm.nih.gov/SNP/). The selected sgHTT with their associated PAM are represented by squares (see SEQ ID NO: 4-7, 13-39). CAG expansion and initiation of the translation (ATG) are also represented. (C) Schematic representation of strategies used for mHTT repair by HR. Donor templates with (upper panel) or without TSS (lower panel) are indicated. In both cases, the maximum size of the homologous sequence is 4.8 kb to be encapsidated in a promoter-less AAV.

FIG. 3: Optimization of Cas9 and sgRNA delivery in vitro. (A) HEK-293T cells were infected with increasing amounts of LV-TARGET (MOI 1, 10, 50 and 100) to produce HEK-TARGET cells. FACS analysis of mCherry fluorescence was used to select the corresponding cell populations. The number of integrated provirus was determined by qPCR on genomic DNA. The four cell populations contain on average 12, 32, 52 and 91 integrated target sequences, respectively. (B) Equal amounts of LV-hCas9 and LV-sgTARGET were incubated on 300′000 cells of the 4 populations. Genomic DNA was extracted 3, 7, 14 and 21 days post-infection to perform surveyor mutation detection assay. Results show that the amount of DSB increases with time in all the populations (Time effect, factorial ANOVA, p<0.01). Cells containing low numbers of target sequences reach a maximal DSB earlier than cells containing higher copies of target sequence, (MOI effect, factorial ANOVA, p<0.01). Results are presented as mean±SEM. (C) HR was evaluated in HEK-TARGET cells (MOI 12 and 32) by transient transfection of plasmids encoding hCas9, the sgTARGET and the homologous donor DNA. Genomic DNA was extracted 3 and 7 days post-transfection to perform surveyor mutation detection assay and NcoI RFLP to evaluate the percent of HR. Surveyor analysis reveal up to 45% of DSB at 7 days post-transfection (data not shown). NcoI digestion displays an efficient HR at the expected locus 3 days post-transfection. Efficiency of HR increased with time to reach up to 37.5% at 7 days post-transfection. Results are presented as mean±SEM. ** p<0.01, *** p<0.001. (D) Results obtained for HR experiments were confirmed by western blots using anti-mCherry antibody. The presence of a band around 54 kDa corresponding to the molecular weight of mCherry-GFP fusion protein is observed in treated samples. Actin was used as a control for normalization. HR1-4: four replicates transfected with plasmids expressing hCas9, sgTARGET and the donor DNA. AAV: cells transfected with the donor DNA and an empty plasmid. NT: untransfected cells. (E) Additive effects of multiple sgRNA targeting a reporter gene. Plasmids encoding GFP, hCas9, H1-sgGFP1 (CRISPR-sgGFP) or H1-sgGFP1-U6-sgGFP2-7SK-sgGFP3 (CRISPR-poly-sgGFP) were co-transfected in HEK-293T cells. Cells co-transfected without the hCas9 plasmid (sgGFP and poly-sgGFP) were used as negative controls. Genomic DNA was extracted 3 days post-transfection, PCR-amplified and used for Surveyor analysis. Quantifications show an increase in the rate of NHEJ formation at the target locus with the combination of the three sgGFP targeting the same region (respectively 81% and 52% with only the sgGFP1).

FIG. 4: Reporter gene editing in neurons and astrocytes in vitro and in vivo. (A) Mouse Ell cortical primary neurons were infected by an LV expressing GFP at day in vitro 1 (DIV1). A second infection was performed at DIV4 with LV expressing hCas9 and LV expressing Tomato-sgGFP1 (CRISPR-GFP), LV-hCas9 and LV expressing mCherry reporter protein (Cas9-Cherry) or LV-Tomato-sgGFP1 alone (sgGFP). Genomic DNA was extracted 3 weeks post-infection with the CRISPR system (DIV25) and subjected to Surveyor assay. Results show around 45% of NHEJ formation at the target site in the CRISPR-GFP group whereas less than 5% of NHEJ was observed in control samples. NI: non-infected neurons. (B) Mouse P1 cortical primary astrocytes were infected by an LV expressing GFP at DIV7. A second infection was performed at DIV10 with LV expressing hCas9 and LV expressing Tomato-sgGFP1 (CRISPR-GFP), LV-hCas9 and LV expressing mCherry reporter protein (Cas9-Cherry) or LV-Tomato-sgGFP1 alone (sgGFP). Genomic DNA was extracted 3 weeks post-infection with the CRISPR system (DIV25) and subjected to Surveyor assay. Results show around 30% of NHEJ formation at the target site in the CRISPR-GFP group whereas less than 5% of NHEJ was observed in control samples. (C) Mouse Ell cortical primary neurons were infected by an LV expressing GFP at DIV1. A second infection was performed at DIV4 with LV expressing hCas9 and LV expressing Tomato-sgGFP1 (CRISPR-GFP) or LV-Tomato-sgGFP1 alone (sgGFP). Neurons were fixed with 4% PFA 3 weeks post-infection with the CRISPR system (DIV25). Microscope acquisitions show numerous GFP-positive neurons in the sgGFP group whereas few GFP-positive cells were observed in the CRISPR-GFP group, showing that GFP disruption leads to the loss of GFP fluorescence. (D) Episomal DNA editing was evaluated in the mouse brain with AAV vectors. AAV expressing GFP and LV expressing hCas9 and Tomato-sgGFP1 (CRISPR-GFP, right panel) or AAV-GFP alone (left panel) were co-injected in the mouse striatum. Brains were fixed 3 weeks post-infection. Direct fluorescence reveals a strong GFP loss at the Tomato-positive area in the CRISPR-GFP group. (E) Genomic DNA editing was evaluated in vivo with LV. LV-GFP, LV-hCas9 and LV-Tomato-sgGFP1 (CRISPR-GFP) or LV-GFP and LV-Tomato-sgGFP1 (sgGFP) were co-injected in the mouse striatum. Brains were fixed with 4% PFA 3 weeks post-infection. Mosaic pictures show a high GFP loss in the Tomato-positive area in the CRISPR-GFP group whereas Tomato-positive-GFP-positive neurons were observed in the sgGFP group. Cell-by-cell GFP fluorescence intensity quantification reveals around 95% of GFP loss in the CRISPR-GFP samples compared to the controls. (F) Genomic DNA of the Tomato-positive area was extracted from mouse injected with the same experimental design as in FIG. 5E. The target sequence was PCR-amplified and subjected to Surveyor assay. Quantifications show around 50% of NHEJ formation at the target sequence in CRISPR-GFP group whereas no GFP disruption was observed in sgGFP samples. (G) LV expressing hCas9 under the gfaABC1D(B)₃ promoter (astrocytic) and LV-Tomato-sgGFP1 (CRISPR-GFP) were injected into the striatum of BAC GLT1-eGFP mice expressing eGFP in astrocytes. Mice injected only with the LV-Tomato-sgGFP1 were used as negative controls (sgGFP). Brains were processed 3 weeks post-infection. Mosaic acquisitions reveal a strong loss of eGFP in astrocytes on the CRISPR-GFP group compared to the sgGFP samples.

FIG. 5: Impact of V5 tag on hCas9 expression and functionality. (A) HEK-293T cells were co-transfected with a plasmid expressing nuclear GFP (GFPnuc) and an AAV plasmid expressing hCas9-V5 under the CBA promoter (AAV-CBA-Cas9-V5) or an AAV plasmid expressing hCas9-V5 under the PGK promoter (AAV-PGK-Cas9-V5) or an LV plasmid expressing hCas9-V5 under the CMV promoter (SIN-CMV-Cas9-V5) or an LV plasmid expressing hCas9-V5 under the PGK promoter (SIN-PGK-Cas9-V5). As positive control for the V5 immunostaining, we used a plasmid expressing a Tau-V5 protein (SIN-PGK-Tau-V5). Cells were fixed 3 days post-transfection and an immunostaining against V5-tag was performed. Pictures reveal that all plasmids encoding hCas9 are expressed. In addition, the majority of the staining was observed in the cytoplasm with few staining in the nucleus, meaning that the addition of the V5-tag reduced the incorporation of hCas9 into the nucleus. (B) Efficiency of hCas9-V5 was determined in HEK-293T cells. Cells were co-transfected with plasmids coding for sgTARGET, TARGET sequence and hCas9 (Cas9 group) or hCas9-V5 (hCas9-V5) group. Genomic DNA was extracted 3 days post-transfection and indels formation was analyzed with the Surveyor assay. Results show that both hCas9 and hCas9-V5 efficiently disrupt target sequence. (C) Efficiency of hCas9-V5 was evaluated by infection in mouse E17 cortical primary neurons. Neurons were infected by an LV expressing GFP at DIV1. A second infection was performed at DIV4 with LV expressing hCas9 (Cas9 group) or LV-hCas9-V5 (Cas9-V5 group) and LV expressing Tomato-sgGFP1. Neurons infected only with LV-sgGFP1 (sgGFP) were used as negative controls. Genomic DNA was extracted 4 weeks post-infection with the CRISPR system (DIV31) and subjected to Surveyor assay. Results show equal efficiency for both hCas9 and hCas9-V5 in primary neurons.

FIG. 6: Editing of WT HTT gene in HEK-293T cells. (A-B) Plasmids encoding hCas9 and sgHTT1 (downstream of the ATG, conserved in mouse and human, panel A) or sgHTT3 (panel B) were transfected in HEK-293T cells for a period of 3 and 7 days (only for sgHTT1). Genomic DNA was extracted for surveyor mutation detection analysis. Transfections with sgHTT1 or hCas9 only were used as negative controls for sgHTT1 experiment whereas co-transfection of plasmids encoding hCas9 and sgGFP1 (CRISPR-GFP) was used as control for sgHTT3 experiment. Results show a mean of 45% of DSB formation at the HTT locus at both time points for sgHTT1, whereas a lower efficiency (mean of 21%) was observed at the sgHTT3 target site. (C) Plasmids encoding hCas9, sgHTT1 and the first 171 amino-acids of the human HTT with 82 CAG fused to the GFP (Htt171-82Q-GFP) were transfected in HEK-293T cells for 3 and 7 days. Total proteins were extracted and the amount of mutant HTT was evaluated by western blot with an anti-HTT antibody. Results shown a strong loss of mutant HTT in samples treated with hCas9 and the sgHTT1 when compared with control samples. Actin was used as control protein for normalization. Results are presented as mean±SEM. * p<0.05, *** p<0.001. (D) Analysis 3 and 7 days post-transfection indicates that control cells (Control panels) transfected with hCas9, Htt171-82Q-GFP and an sgRNA (no homology for either the target plasmid) have a high number of GFP-positive aggregates whereas HEK-293T cells transfected with hCas9, sgHTT1 and Htt171-82Q-GFP (CRISPR panels) have a reduced number GFP-positive aggregates of less intense GFP-signal. Scale bar=200 nm.

FIG. 7: Mutant HTT disruption in primary neurons and in the striatum. (A) Mouse E17 cortical primary neurons were infected by LV-hCas9 and LV-sgHTT1 (CRISPR-HTT1), LV-hCas9 and LV-sgGFP1 (CRISPR-GFP) or LV-sgHTT1 (sgHTT1) at DIV1. A second infection was performed at DIV4 with LV expressing the first 171 amino acid of the human HTT with 82 CAG (LV-171HTT-82Q). Genomic DNA was extracted 3 weeks post-infection with the CRISPR system (DIV25). Primers specific for the human HTT were used to amplify the mHTT. Surveyor analysis shows around 35% of human HTT disruption in the CRISPR-HTT1 group (left part of the graph). Because the sgHTT1 has full homology for the mouse HTT except for the first nucleotide (5′ end of the crRNA), mouse-specific primers were used to amplify the endogenous HTT for surveyor assay. Results show that the sgHTT1 is able to induce mouse HTT disruption at the same efficiency than human HTT. (B) Mouse Ell cortical primary neurons were infected by LV-hCas9 and LV-sgHTT1 (CRISPR-HTT1) or LV-hCas9 and LV-sgGFP1 (CRISPR-GFP) at DIV1. A second infection was performed at DIV4 with LV expressing the first 171 amino acid of the human HTT with 82 CAG (LV-171HTT-82Q). Neurons were fixed at DIV25 with 4% PFA and an immunostaining against the mutant HTT was performed. Microscope acquisitions in the CRISPR-GFP group reveal numerous large mHTT aggregates (light gray arrow heads), which are mainly localized in the nucleus and cytoplasm of neurons, and small mHTT aggregates (dark gray arrow heads), which are mainly localized in neuritis. A strong loss of large and small mHTT aggregates is observed in the CRISPR-HTT group as a result of mHTT disruption. (C) LV-171HTT-82Q, LV-hCas9 and LV-sgHTT1-mCherry (CRISPR-HTT) were co-injected into the striatum of C57Bl/6 mice. As negative controls, LV-171HTT-82Q, LV-hCas9 and LV-Tomato-sgGFP1 (CRISPR-GFP) were used. Brains were processed 4 weeks post-injection and stained with an anti-mHTT antibody. Pictures reveal a strong loss of mHTT-positive aggregates in the CRISPR-HTT group compared with the CRISPR-GFP group.

### BRIEF DESCRIPTION OF THE SEQUENCES

SEQ ID NO: 1 corresponds to the 82 nucleotides of the artificial chimeric tracrRNA containing the poly-T signal: 5′-GTTTTAGAGCTAGAAATAGCAAGTTAAAATAAGGCTAGTCCGTTATCAACTTGAA AAAGTGGCACCGAGTCGGTGCTTTTTT-3′

SEQ ID NO: 2 corresponds to the 20 nucleotides of the artificial sgTARGET:

5′-GGCTGCTGTCAGGGCTAGCA-3′

SEQ ID NO: 3 corresponds to the 20 nucleotides of the artificial sgGFP:

5′-GAAGTTCGAGGGCGACACCC-3′

SEQ ID NO: 4 corresponds to the 20 nucleotides of the sgHTT1 in exon 1, which is conserved between human, Macaca Fascicularis and Mus Musculus HTT gene with one mismatch at position 1 (G/A):

5′-GACCCTGGAAAAGCTGATGA-3′

SEQ ID NO: 5 corresponds to the 20 nucleotides of the sgHTT2, which is fully conserved between humans, Macaca Fascicularis, Rattus Norvegicus and Mus Musculus HTT gene:

5′-GTGGATGACATAATGCTTTT-3′

SEQ ID NO: 6 corresponds to the 20 nucleotides of the sgHTT3 between exon 1 and between intron 1, which is conserved between humans, Macaca Fascicularis, Rattus Norvegicus and Mus Musculus HTT gene with one mismatch at position 4 (G/A):

5′-GGAGCCGCTGCACCGACCGT-3′

SEQ ID NO: 7 corresponds to the 20 nucleotides of the sgHTT4, which is human specific with SNP rs13102260 at position 20 (G/A underline) for allele-specific approaches:

5′-TGGGACGCAAGGCGCCGTGG-3′

SEQ ID NO: 8 corresponds to the human codon-optimized Cas9 fused to one nuclear localization signal (NLS coding for: PKKKRKV) at the C-terminus (sequence: https://www.addgene.org/41815, (17)).

SEQ ID NO: 9 corresponds the artificial HR sequence used in the example 2 containing the end of the mCherry, the corrected target site and the GFP sequence.

SEQ ID NO: 10 corresponds to the 20 nucleotides of the artificial sgGFP2:

5′-GCTGAAGGGCATCGACTTCA-3′

SEQ ID NO: 11 corresponds to the 20 nucleotides of the artificial sgGFP3:

5′-CAACTACAAGACCCGCGCCG-3′

SEQ ID NO: 12 corresponds to the lentiviral vector SIN-cPPT-H1-sgGFP1-U6-sgGFP2-7SK-sgGFP3-PGK-mCherry-WPRE for the expression of multiple sgRNA under the control polymerase III promoters (H1, U6, 7SK) and containing a mCherry reporter gene to visualize infected cells.

SEQ ID NO: 13 corresponds to the 20 nucleotides of the artificial sgHTT24, which is human-specific and containing the human SNP rs9993542 at position 19 (C/T) for allele-specific approaches:

5′-GGTAATTTGTGTTTGTGTGC-3′

The sgHTT24 was designed on the complementary strand of the HTT gene, as a consequence the polymorphism of the human SNP rs9993542 at position 19 corresponds to the complementary sequence (G/A underline).

SEQ ID NO: 14 corresponds to the 20 nucleotides of the artificial sgHTT25, which is human specific with SNP rs762855 at position 18 (A/G) for allele-specific approaches

5′-GACAGCAGAGAAACAGCTGT-3′

The sgHTT25 was designed on the complementary strand of the HTT gene, as a consequence the polymorphism of the human SNP rs762855 at position 18 corresponds to the complementary sequence (T/C underline).

SEQ ID NO: 15 corresponds to the 20 nucleotides of the artificial sgHTT5, which is conserved between human and Macaca Fascicularis with one mismatch at the 11^{th} position from 5′ (A/G) and containing the human SNP rs762855 at position 11 (A/G) for allele-specific approaches

5′-GAGAAACAGCTGTTAGTTCC-3′

The sgHTT5 was designed on the complementary strand of the HTT gene, as a consequence the polymorphism of the human SNP rs762855 at position 11 corresponds to the complementary sequence (T/C underline).

SEQ ID NO: 16 corresponds to the 20 nucleotides of the artificial sgHTT26, which is human specific with SNP rs9996199 at position 19 (C/G) for allele-specific approaches

5′-TGGCTAAAGTAGGCTTTACT-3′

SEQ ID NO: 17 corresponds to the 20 nucleotides of the artificial sgHTT6, which is human specific with SNP rs28431418 at position 19 (T/C underline) for allele-specific approaches

5′-CAGGGCTGTCCGGGTGAGTA-3′

SEQ ID NO: 18 corresponds to the 20 nucleotides of the artificial sgHTT7, which is human specific with SNP rs28431418 at position 13 (T/C underline) for allele-specific approaches

5′-TGTCCGGGTGAGTATGGCTC-3′

SEQ ID NO: 19 corresponds to the 20 nucleotides of the artificial sgHTT27, which is human specific with SNP rs2857935 at position 20 (G/C/T) for allele-specific approaches

5′-AGGCCCATGCGGAAAGGATC-3′

The sgHTT27 was designed on the complementary strand of the HTT gene, as a consequence the polymorphism of the human SNP rs2857935 at position 20 corresponds to the complementary sequence (C/G/A underline).

SEQ ID NO: 20 corresponds to the 20 nucleotides of the artificial sgHTT8, which is human specific with SNP rs2857935 at position 8 (G/C/T underline) for allele-specific approaches

5′-GGCGGGGGATCCTTTCCGCA-3′

SEQ ID NO: 21 corresponds to the 20 nucleotides of the artificial sgHTT10, which is human specific with SNP rs13122415 at position 17 (C/G) for allele-specific approaches

5′-GGGGCTCAACGGAGAGGGGA-5′

The sgHTT10 was designed on the complementary strand of the HTT gene, as a consequence the polymorphism of the human SNP rs13122415 at position 17 corresponds to the complementary sequence (G/C underline)

SEQ ID NO: 22 corresponds to the 20 nucleotides of the artificial sgHTT28, which is human specific with SNP rs13132932 at position 19 (A/G underline) for allele-specific approaches

5′-GGCAGAACCTGCGGGGGCAG-3′

SEQ ID NO: 23 corresponds to the 20 nucleotides of the artificial sgHTT9, which is human specific with SNP rs13132932 at position 15 (A/G underline) for allele-specific approaches

5′-GAACCTGCGGGGGCAGGGGC-3′

SEQ ID NO: 24 corresponds to the 20 nucleotides of the artificial sgHTT14, which is human specific with SNP rs34045730 at position 17 (A/T underline) for allele-specific approaches

5′-ATTTTACCAGTATTCCAGTC-3′

SEQ ID NO: 25 corresponds to the 20 nucleotides of the artificial sgHTT15, which is human specific with SNP rs28656215 at position 20 (C/T underline) for allele-specific approaches

5′-CTGTGTGTCGAGTGTACAGT-3′

SEQ ID NO: 26 corresponds to the 20 nucleotides of the artificial sgHTT16, which is human specific with SNP rs3856973 at position 20 (G/A) for allele-specific approaches

5′-ATAAAAATAAGTTAACACTC-3′

The sgHTT16 was designed on the complementary strand of the HTT gene, as a consequence the polymorphism of the human SNP rs3856973 at position 20 corresponds to the complementary sequence (C/T underline).

SEQ ID NO: 27 corresponds to the 20 nucleotides of the artificial sgHTT17, which is human specific with SNP rs57666989 at position 14 (C/T) for allele-specific approaches

5′-GCAGGAGAATGGCGTGAACT-3′

The sgHTT17 was designed on the complementary strand of the HTT gene, as a consequence the polymorphism of the human SNP rs57666989 at position 14 corresponds to the complementary sequence (G/A underline).

SEQ ID NO: 28 corresponds to the 20 nucleotides of the artificial sgHTT19, which is fully conserved between human and Macaca Fascicularis with human SNP rs61792477 at position 12 (C/T underline) for allele-specific approaches

5′-ATAATATAGTACGGCCACTC-3′

SEQ ID NO: 29 corresponds to the 20 nucleotides of the artificial sgHTT20, which is human specific with SNP rs28867436 at position 19 (A/G underline) for allele-specific approaches

5′-CATAAAACTGAACATACCGT-3′

SEQ ID NO: 30 corresponds to the 20 nucleotides of the artificial sgHTT21, which is human specific with SNP rs2285087 at position 15 (A/G underline) for allele-specific approaches

5′-AGCAATTGGGCAACATCGTG-3′

SEQ ID NO: 31 corresponds to the 20 nucleotides of the artificial sgHTT30, which is human specific with SNP rs2285087 at position 17 (A/G) for allele-specific approaches

5′-AGACGGGGCCTCACGATGTT-3′

The sgHTT30 was designed on the complementary strand of the HTT gene, as a consequence the polymorphism of the human SNP rs2285087 at position 17 corresponds to the complementary sequence (T/C underline).

SEQ ID NO: 32 corresponds to the 20 nucleotides of the artificial sgHTT22, which is human specific with SNP rs2285086 at position 20 (A/G) for allele-specific approaches

5′-AGAAGGTGCTGCTAGTTCAT-3′

The sgHTT22 was designed on the complementary strand of the HTT gene, as a consequence the polymorphism of the human SNP rs2285086 at position 20 corresponds to the complementary sequence (T/C underline).

SEQ ID NO: 33 corresponds to the 20 nucleotides of the artificial sgHTT18, which is human specific with SNP rs61792473 at position 12 (C/T) and rs61792474 at position 14 (C/T) for allele-specific approaches

5′-TGTGTGTGTGTGTGTATATT-3′

The sgHTT18 was designed on the complementary strand of the HTT gene, as a consequence the polymorphism of the human SNP SNP rs61792473 at position 12 and rs61792474 at position 14 corresponds to the complementary sequence (G/A underline).

SEQ ID NO: 34 corresponds to the 20 nucleotides of the artificial sgHTT13, which is human specific with SNP rs58870770 at position 19 (−/A) (−=deletion of one nucleotide) for allele-specific approaches

5′-GTTGTGGTTGCCAAGTAAAG-3′

SEQ ID NO: 35 corresponds to the 20 nucleotides of the artificial sgHTT12, which is human specific with SNP rs150577220 at position 14 (−/A) (−=deletion of one nucleotide) for allele-specific approaches

5′-GGAGTGGCGGGGCAGGGGGG-3′

SEQ ID NO: 36: corresponds to the 20 nucleotides of the human-specific artificial sgHTT31:

5′-CGGGACGGGTCCAAGATGGA-3′

SEQ ID NO 37: corresponds to the 20 nucleotides of the human-specific artificial sgHTT35, which is fully conserved human and Macaca Fascicularis

5′-GGGCTGTCAATCATGCTGGC-3′

SEQ ID NO: 38: corresponds to the 20 nucleotides of the artificial sgHTT33, which is fully conserved between human and Macaca Fascicularis:

5′-CTTTTCCAGGGTCGCCATGG-3′

SEQ ID NO: 39: corresponds to the 20 nucleotides of the artificial sgHTT34, which is fully conserved between human and Macaca Fascicularis:

5′-GACAATATGTGAAAACATAG-3′

SEQ ID NO: 40 corresponds to the 20 nucleotides of the artificial sgCas9:

5′-AATGGAGTACTTCTTGTCCA-3′

SEQ ID NO: 41 Cas9-V5 corresponds to the human codon-optimized Cas9 fused to one nuclear localization signal and V5 epitope at the C-terminus of Cas9 to visualize the protein.

SEQ ID NO: 42 human HTT with the TSS corresponds to one exogenous DNA template used for HR.

SEQ ID NO: 43 human HTT HR without the TSS corresponds to one exogenous DNA template used for HR.

### DETAILED DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components.

As used in the specification and claims, the singular forms “a”, “an” and “the” include plural references unless the context clearly dictates otherwise.

As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “an effective amount” refers to an amount necessary to obtain a physiological effect. The physiological effect may be achieved by one application dose or by repeated applications.

The dosage administered may, of course, vary depending upon known factors, such as the physiological characteristics of the particular composition; the age, health and weight of the subject; the nature and extent of the symptoms; the kind of concurrent treatment; the frequency of treatment; and the effect desired and can be adjusted by a person skilled in the art.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g. phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e. the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase).

The term “Tag” refers to short amino acid sequences constituting an epitope recognized by an antibody. Epitope tags are added at the N- or C-terminus of a protein to facilitate visualization by immunohistochemistry. Epitope tags include, for example, one or more copies of FLAG, His, myc, Tap, HA, V5 or any detectable amino acid sequence. Cas9 has been previously fused to FLAG tags however; Applicants preferably use the V5 tag to localize Cas9 protein in the present experiments. “Expression tags” include sequences that encode reporters that may be operably linked to a desired gene sequence in order to monitor expression of the gene of interest.

“The CRISPR” (Clustered Regularly Interspaced Short Palindromic Repeats)/Cas9 (CRISPR Associated) nuclease system is a recently engineered nuclease system based on a bacterial system that can be used for genome engineering. It is based on part of the adaptive immune response of many bacteria and archea. When a virus or plasmid invades a bacterium, segments of the invader's DNA are converted into CRISPR RNAs (crRNA) by the ‘immune’ response. This crRNA then associates, through a region of partial complementarity, with another type of RNA called tracrRNA to guide the Cas9 nuclease to a region homologous to the crRNA in the target DNA called a “protospacer.” Cas9 cleaves the DNA to generate blunt ends at the DSB at sites specified by a 20-nucleotide guide sequence contained within the crRNA transcript. Cas9 requires both the crRNA and the tracrRNA for site-specific DNA recognition and cleavage. This system has now been engineered such that the crRNA and tracrRNA can be combined into one molecule (the “single guide RNA”), and the crRNA equivalent portion of the single guide RNA can be engineered to guide the Cas9 nuclease to target any desired sequence (9, 18). Thus, the CRISPR/Cas9 system can be engineered to create a DSB at a desired target in a genome, and repair of the DSB can be influenced by the use of repair inhibitors to cause an increase in error prone repair.

As used herein the term “selectivity of an sgRNA” relates to the ability for an sgRNA sequence and Cas9 complex, to recognize a particular allele of a target gene (i.e. mutant vs WT allele of HTT).

The term “specificity of an sgRNA” relates to the ability for a given sgRNA sequence and Cas9 complex to recognize a given target i.e., the HTT gene.

The term “haplotype” relates to the ordered, linear combination of polymorphisms (e.g., SNPs) in the sequence of each form of a gene (on individual chromosomes) that exists in the population.

The term “small insertions-deletions (indels)” relates to small insertion or deletions in genomic DNA due to cellular DNA repair following DSB by non-homologous end joining (NHEJ).

The term “allele” relates to a particular form of a genetic locus, distinguished from other forms by its specific nucleotide sequence.

A “Single Nucleotide Polymorphism” (SNP) refers to a single base (nucleotide) difference in a specific location in the DNA sequence among individuals in a population. A subset of SNPs gives rise to changes in the encoded amino acid sequence; these are referred to as coding SNPs, or cSNPs.

The term “allele-specific” when used in reference to nucleic acid sequences, such as oligonucleotides, primers, sgRNA, means that a particular position of the nucleic acid sequence is complementary with an allele of a target polynucleotide sequence. Allele-specific sgRNA are capable of discriminating between different alleles of a target polynucleotide.

The term “vector”, as used herein, refers to a DNA or RNA molecule such as a plasmid, virus or other vehicle, which contains one or more heterologous or recombinant DNA sequences and is designed for transfer between different host cells. The terms “expression vector” and “gene therapy vector” refer to any vector that is effective to incorporate and express heterologous DNA fragments in a cell. A cloning or expression vector may comprise additional elements, for example, post-transcriptional regulatory elements. Any suitable vector can be employed that is effective for introduction of nucleic acids into cells, e.g. a viral vector. The terms “heterologous DNA” refer to a “heterologous coding or non-coding sequence” or a “transgene”.

The term “homology” between two sequences is determined by sequence identity. If two sequences, which are to be compared with each other differ in length, sequence identity preferably relates to the percentage of the nucleotide residues of the shorter sequence which are identical with the nucleotide residues of the longer sequence. Sequence identity can be determined conventionally with the use of computer programs such as the Bestfit program (Wisconsin Sequence Analysis Package, Version 8 for Unix, Genetics Computer Group, University Research Park, 575 Science Drive Madison, Wis. 53711). Bestfit utilizes the local homology algorithm of Smith and Waterman, Advances in Applied Mathematics 2 (1981), 482-489, in order to find the segment having the highest sequence identity between two sequences. When using Bestfit or another sequence alignment program to determine whether a particular sequence has for instance 95% identity with a reference sequence of the present invention, the parameters are preferably so adjusted that the percentage of identity is calculated over the entire length of the reference sequence and that homology gaps of up to 5% of the total number of the nucleotides in the reference sequence are permitted. When using Bestfit, the so-called optional parameters are preferably left at their preset (“default”) values. The deviations appearing in the comparison between a given sequence and the above-described sequences of the invention may be caused for instance by addition, deletion, substitution, insertion or recombination. Such a sequence comparison can preferably also be carried out with the program “fasta20u66” (version 2.0u66, September 1998 by William R. Pearson and the University of Virginia; see also W. R. Pearson (1990), Methods in Enzymology 183, 63-98, appended examples and http://workbench.sdsc.edu/). For this purpose, the “default” parameter settings may be used.

It is one object of the invention to provide a kit for the treatment of Huntington's disease (HD) for allele or non-allele-specific huntingtin (HTT) gene editing or repair comprising:
a gene delivery vector consisting of at least one viral vector selected among adeno-associated vector serotypes (AAV) and/or lentiviral vectors (LV);
a Cas9 being human codon-optimized or fused to an epitope tag selected among the group of FLAG, His, myc, Tap, HA, V5. Preferably Cas9 is fused to an epitope tag (e.g V5 tag).
at least one artificial single guide RNA (sgRNA) having a total size from 63-115 nucleotides comprising a tracrRNA sequence of 48-85 nucleotides long (preferably 82 bp) and a crRNA sequence of 15-30 nucleotides (preferably 20 bp) recognizing the sequence of the HTT gene around the expanded CAG repeat mutation (ENSG00000197386, position of the first CAG repeat in exon 1: 3′074′877-3′074′879), and comprising the region upstream of the HTT gene (position 3066800) up to the beginning of intron 2 (position 3087600), wherein said crRNA sequence binds directly upstream of the required 5′-NGG/NAG-3′ protospacer adjacent motif (PAM) and whereas said crRNA sequence base-pairs with the target HTT sequence and Cas9 mediates a double-stranded break (DSB) 3-4 bp upstream of said PAM.

In particular, the region around the expanded CAG repeat mutation in the HTT gene includes:

the promoter (Ensembl accession number: ENSG00000197386; positions 3066800 to 3074509),

exon 1 of the HTT gene including the 5′UTR the transcription and translation start sites (TSS and ATG; Ensembl accession number: ENSG00000197386: positions 3074510 to 3075088),

intron 1 of the HTT (Ensembl accession number: ENSG00000197386: positions 3075089 to 3086939),

exon 2 (Ensembl accession number: ENSG00000197386: positions 3086940 to 3087023),

and 5′ end on intron 2 (Ensembl accession number: ENSG00000197386: positions 3087024 to 3087600).

Preferably, the gene delivery vector contains various expression cassettes comprising promoters and/or miRNA-regulated system so as to modulate transgene expression.

In a preferred mode of the invention, the AAV is a mutant vector and preferably the AAV-DJ for HR.

In another embodiment, of the invention, the Cas9 is mutated to improve the efficiency and safety. Preferably said mutated Cas9 is a Cas9 nickase or a Cas9-V5.

In another embodiment of the invention the gene delivery vector consist in a single vector or several gene delivery vectors.

According to a preferred mode of the invention, the kit comprises multiple transcriptionally independent sgRNA targeting the HTT gene.

In particular, said artificial sgRNA comprises a sequence (crRNA) recognizing a target site of 15-30 nucleotides, preferably 20 nt, recognizing the HTT gene is human-specific or a conserved sequence between human, rodent and primates with a maximum tolerated number of mismatches between sgRNA and HTT gene of 3 in the first 8 nucleotides of the sgRNA.

According to a preferred mode, said artificial crRNA sequence of 15-30 nucleotides is encoded by the sequences selected among the group of SEQ ID NO: 4-7, SEQ ID NO: 13-39 and/or biological active fragments, variants or mutants thereof being able to recognize the HTT gene.

In another embodiment of the invention, the kit further comprises an exogenous DNA template sharing homology around the DSB target site for homologous recombination (HR). Said exogenous DNA template having at least 95% of homology (preferably 98%) with the homology arms located 10-20 bp away from the double strand break site and with a minimum length of 100 bp and up to 2 kb on both side of the double-stranded break (DSB).

In such embodiment, the exogenous DNA template for HTT HR contains or does not contain the transcriptional start sites (TSS) and said exogenous DNA template may be selected among the group comprising SEQ ID NO: 42-43. Preferably said exogenous DNA template for HR does not contain the TSS such as SEQ ID NO: 43.

It is another object of the invention to provide a kit for the treatment of Huntington's disease, for use in a method for non-allele-specific HTT inactivation of the human (or other species having a conserved sequences) HTT WT (wild type) and mutant alleles by preferentially targeting the region upstream of the HTT gene (position 3066800) up to the beginning of intron 2 (position 3087600). As exemplified in SEQ ID NO: 4-6 and SEQ ID NO: 15, 28, 37-39 or SEQ ID NO 7, 13-14, 16-27, 29-35, if the SNP is homozygous (i.e. WT and mutant HTT alleles have the same sequence at the SNP considered) in the treated HD patients.

Another object of the invention is to provide a kit for the treatment of Huntington's disease, for use in a method for non-allele-specific HTT gene repair based on HR with a DNA template containing a WT HTT sequence, as exemplified in SEQ ID NO: 42-43.

A further object of the invention is to provide a kit for the treatment of Huntington's disease, for use in a method for allele-specific HTT inactivation of the human mutant HTT gene wherein the sgRNA of said kit is capable of recognizing SNP sequences located upstream a PAM along the HTT gene with a high frequency of heterozygosity in the human population at least >5% (according to dbSNP or 1000 Genome Project database), preferably more than >15%, even more than >20% and most preferably >30% according to dbSNP; http://www.ncbi.nlm.nih.gov/projects/SNP/snp_ref.cgi?geneId=3064 or 1000 Genome Project database). This is exemplified for example by SEQ ID NO: 7, SEQ ID NO: 13-38.

The invention also provides a kit for the treatment of Huntington's disease, for use in a method for mutant HTT gene repair based on HR with a DNA template containing a WT HTT sequence wherein the sgRNA of said kit is capable of recognizing SNP sequences located upstream a PAM along the HTT gene with a high frequency of heterozygosity in the human population at least >5% (according to dbSNP or 1000 Genome Project database), preferably more than >15%, even more than >20% and most preferably >30% according to dbSNP; http://www.ncbi.nlm.nih.gov/projects/SNP/snp_ref.cgi?geneId=3064 or 1000 Genome Project database). This is exemplified for example by SEQ ID NO: 7, SEQ ID NO: 13-38.

A frequency of heterozygosity in the human population at least >5%, means that, on average, >5% of the human population will have a different sequence (heterozygous SNP) on the WT and mutant HTT allele at the SNP considered and >5% of the population would be eligible for an allele-specific strategy. As a consequence, the other <95% of the human population will have the same sequence (homozygous SNP) on the WT and mutant HTT alleles and would be eligible for a non-allele specific HTT gene editing. SNP with high frequency of heterozygosity would greatly facilitate allele-specific therapeutic strategies, because a limited number of products need to be developed to treat a great majority of the HD patients.

Permanent expression of Cas9 after target sequence editing could raise potential adverse effects through off-target mutagenesis.

In a preferred embodiment of the invention, the kit for the treatment of Huntington's disease further comprises an additional transcriptionally independent artificial sgRNA capable of self-inactivating Cas9 expression after human HTT editing, wherein said additional transcriptionally independent artificial sgRNA comprises a crRNA sequence of 15-30 nucleotides which recognizes key regions driving Cas9 expression selected among the ATG and/or the promoter driving the expression of Cas9.

In particular said additional transcriptionally independent artificial single guide RNA (sgRNA) has a total size from 63-115 nucleotides, preferably 102 nucleotides, comprising a tracrRNA sequence of 48-85 nucleotides long (preferably 82 bp) and a crRNA sequence of 15-30 nucleotides, preferably 20 nt, recognizing key regions driving Cas9 expression (ATG and/or vector promoter) in order to self-inactivating Cas9 expression after human HTT editing (SEQ ID NO: 40). Weak polymerase III promoters, with for example the 7SK promoter are used to express sgCas9, while strong polymerase III promoters (H1 and U6) are used to express the sgHTT. This ensures a rapid cleavage of the human HTT gene and then a progressive induction of DSB in Cas9 (self-cleavage by the sgCas9). Because of Cas9 gene disruption, the expression of the protein is blocked thus, preventing potential adverse effects associated with long-term and uncontrolled Cas9 expression.

In a yet further embodiment, the kit for the treatment of Huntington's disease according to the present invention, is suitable for use in a method of treating neuronal and glial cells in a patient in need thereof, wherein the gene delivery vector used to deliver said (CRISP system) Cas9 and said at least one artificial single guide RNA (sgRNA) is suitable for a local administration in the human striatum or is engineered to allow diffusion and/or transport from the striatum to the entire of the basal ganglia network of said patient. This is achieved through the combined use of cell-type specific promoters, microRNA regulatory mechanisms, LV pseudotypes with various envelopes or AAV serotypes.

Besides, diffusion and/or transport may be carried out through the use of specific envelopes including the VSV-G-rabies chimeric envelops for the pseudotyping of LV or use of specific AAV serotypes (19, 20).

Preferably the gene delivery vector is capable of targeting neurons and/or glial cells.

Huntington's disease is a neurodegenerative disorder characterized by a selective vulnerability of the striatum in the first stages of the pathology, followed by a more global degeneration throughout the brain, in particular in the basal ganglia network. The invention provides the use of a kit containing viral vectors expressing the CRISPR system after injection into the striatum. Applicants also provide engineered viral vectors with the capacity to diffuse and/or being transported trans-synaptically for infection of cells along the basal ganglia network.

Mutant HTT exerts numerous molecular adverse effects leading to the degeneration of neurons of the striatum (medium-sized spiny neurons or MSN), but also other brain regions at later stages. If neurons are dying, glial cells, in particular astrocytes, are also vulnerable to mHTT and their dysfunctions exacerbate neuronal deficits. Whether treating neurons in HD patients will be sufficient to solve the pathology has not been fully addressed. In the present invention, Applicants provide a kit containing viral vectors expressing the CRISPR system being engineered to target neurons or glial cells or both neuronal and glial cells in HD patients.

Also provided is a pharmaceutical composition comprising the gene delivery vector of the invention in combination with a pharmaceutically acceptable carrier. The above detailed description disclosing the kit of the present invention is incorporated herein by reference in its entirety for a full disclosure of the pharmaceutical composition comprising the gene delivery vector of the present invention. Namely, the pharmaceutical composition comprises the gene delivery vector consisting of at least one viral vector selected among adeno-associated vector serotypes (AAV) and/or lentiviral vectors (LV);

a Cas9 being human codon-optimized or fused to an epitope tag selected among the group of FLAG, His, myc, Tap, HA, V5;

at least one artificial single guide RNA (sgRNA) having a total size from 63-115 nucleotides comprising a tracrRNA sequence of 48-85 nucleotides long and a crRNA sequence of 15-30 nucleotides recognizing the sequence of the HTT gene around the expanded CAG repeat mutation (ENSG00000197386, position of the first CAG repeat in exon 1: 3′074′877-3′074′879), and comprising the region upstream of the HTT gene (position 3066800) up to the beginning of intron 2 (position 3087600), wherein said crRNA sequence binds directly upstream of the required 5′-NGG/NAG-3′ protospacer adjacent motif (PAM) and whereas said crRNA sequence base-pairs with the target HTT sequence and Cas9 mediates a double-stranded break (DSB) 3-4 bp upstream of said PAM.

Preferably, the pharmaceutical composition of the invention further comprises pharmaceutically acceptable diluents, or excipients that are well known to the skilled in the art.

Applicants propose to use gene editing with the CRISPR system approach for HD. It concerns a new therapy for a fatal neurodegenerative disorder with no available treatment. This represents the first attempt of in vivo gene editing and repair for a CNS disorder.

Contrary to WO 2014/093701 (Feng Zhang, MIT, USA), if a gene editing strategy has been proposed for HD, the target sequence is limited to:

1) a mutant HTT allele specific guide RNA: “nucleotide sequences unique to the mutant HTT allele (CAG repeats) and its use to design guide RNA. Authors ensure that the mutant base is located within the last 9 bp of the guide RNA (which Authors have ascertained it has the ability to discriminate between single DNA base mismatches between the target size and the guide RNA)” or alternatively

2) “Authors use Cas9 nickases in combination with pairs of guide RNAs to generate DNA double strand breaks with defined overhangs. It is stated that when two pairs of guide RNAs are used, it is possible to excise an intervening DNA fragment”.

The present invention is based on entirely new and innovative strategies for allele or non-allele-specific HTT editing and which is also capable of gene repair strategies targeting the promoter, transcription and/or translation start sites and/or SNP in the HTT gene. HTT gene disruption based on sgRNA targeting the CAG expansion or double-strand break with overhang (2-4 sgRNA and Cas nickase) are not considered in the present invention.

Applicants strategy is not based on repressors to inhibit mHTT expression (WO 2013/130824 & Garriga-Canut et al. 2012 (7)) but will address the possibility to perform gene editing to inactivate mutant HTT and DNA repair to restore WT HTT expression using DNA-targeted nucleases. In particular, one will use the recently described CRISPR system to induce mHTT double-strand break for permanent mHTT knock-out or repair.

One important prerequisite for the development of in vivo gene repair strategy in the CNS is an efficient delivery system. Applicants have integrated the CRISPR system in lentiviral vectors (LV expressing Cas9 and the sgRNA) and adeno-associated virus (AAV containing the donor DNA for HR or expressing Cas9 and the sgRNA), which have been widely used in vitro but also for in vivo gene delivery in the CNS.

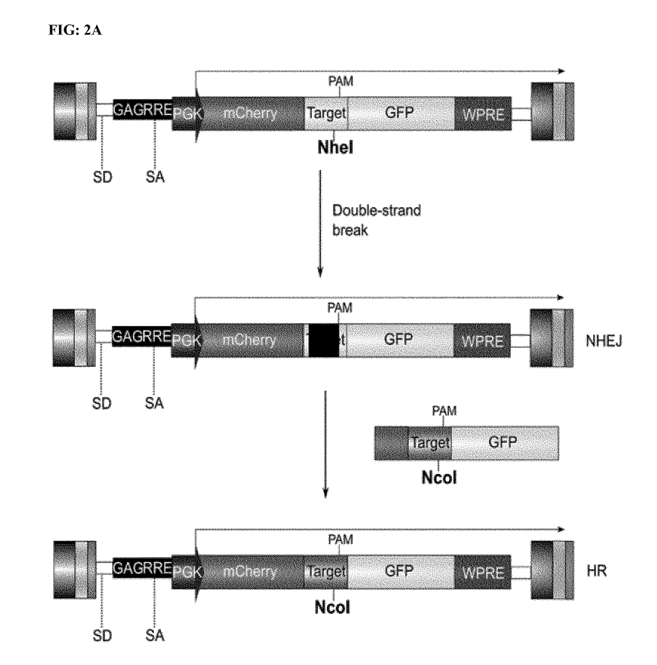

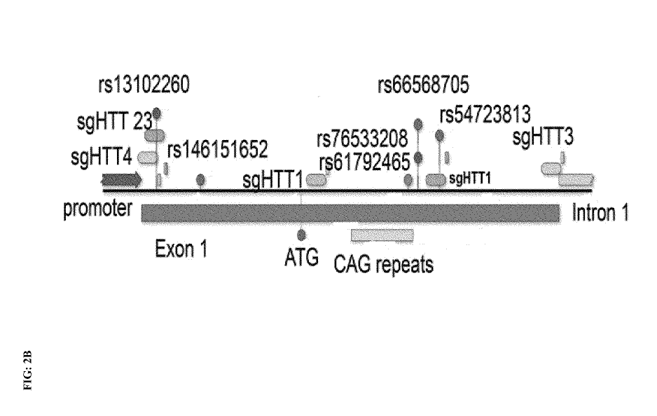

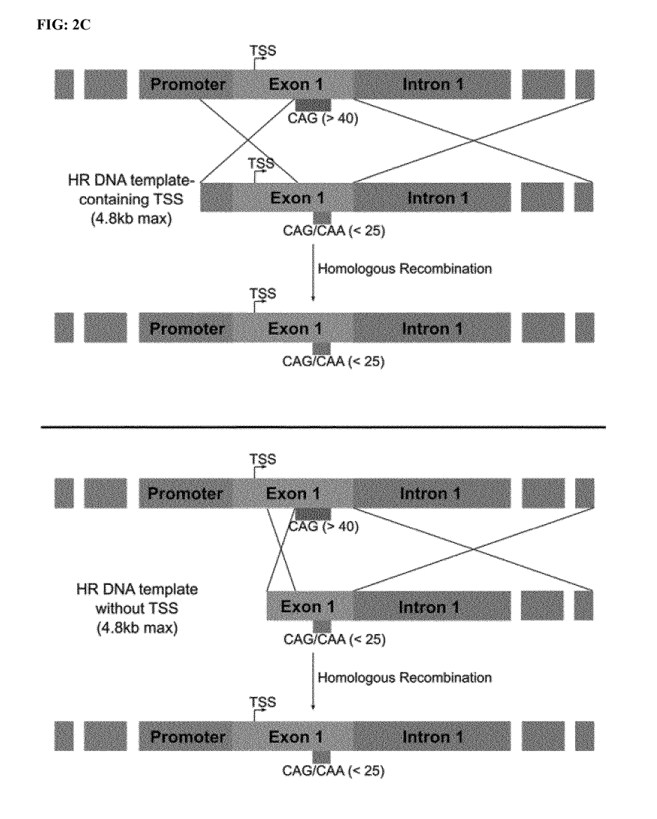

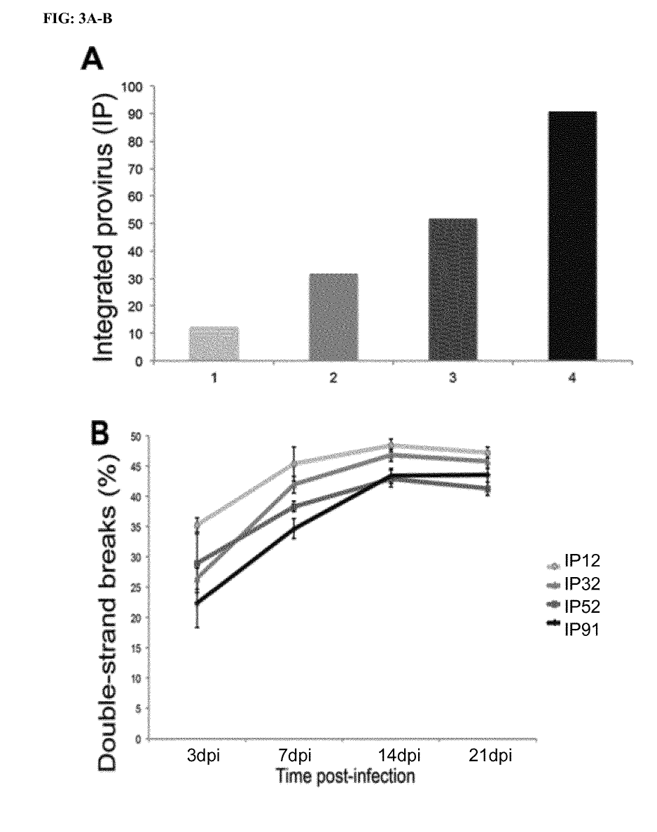

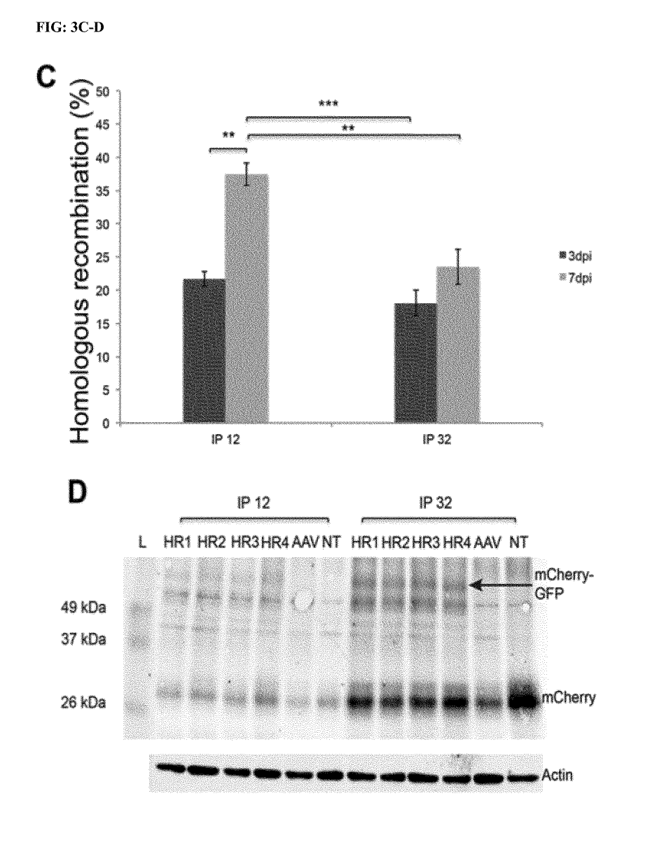

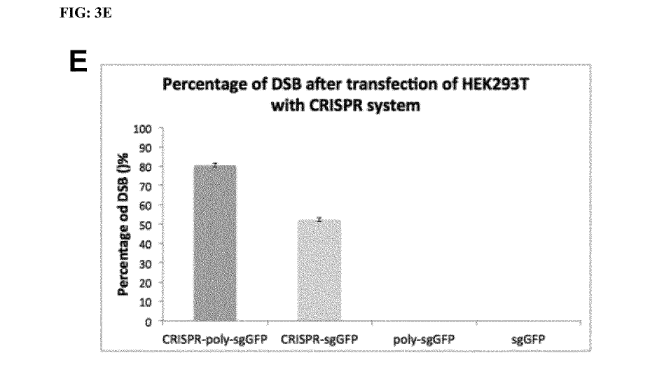

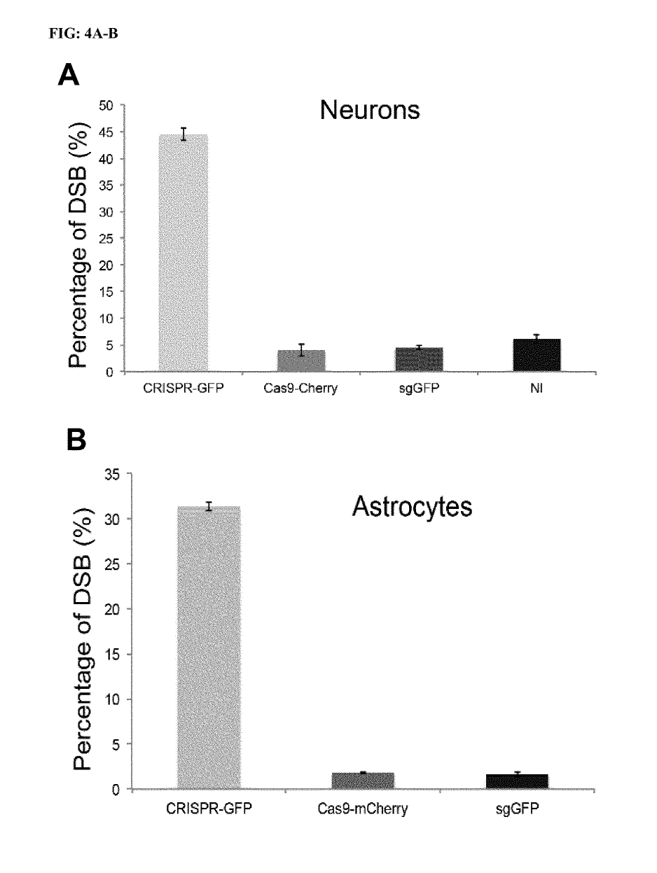

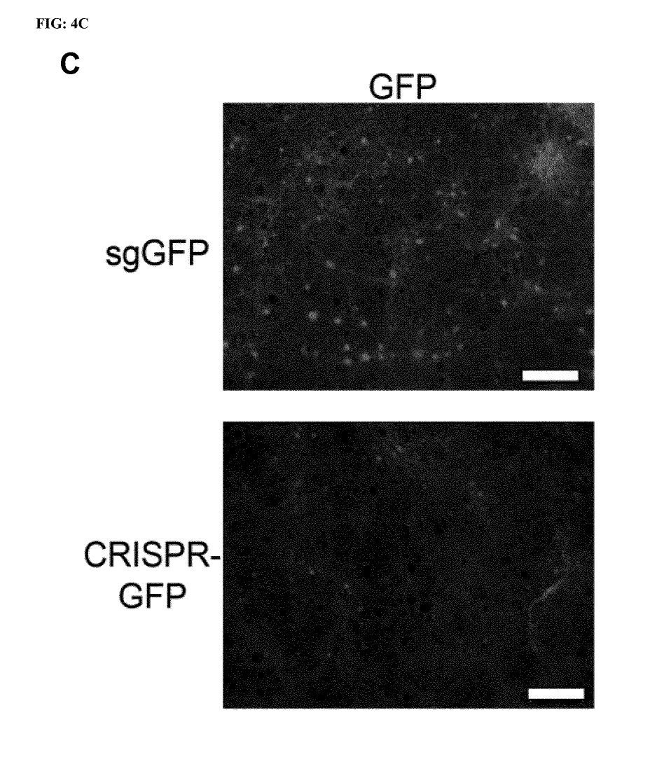

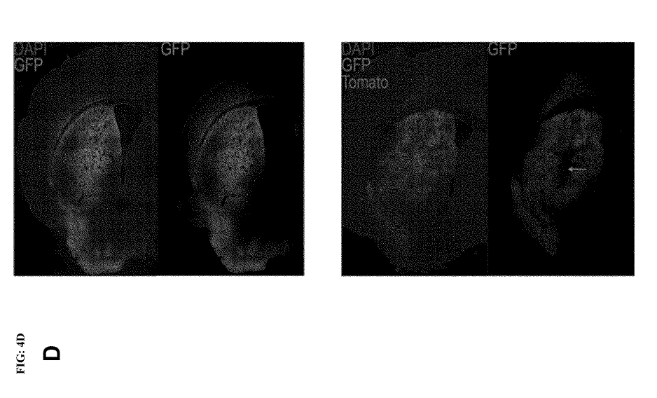

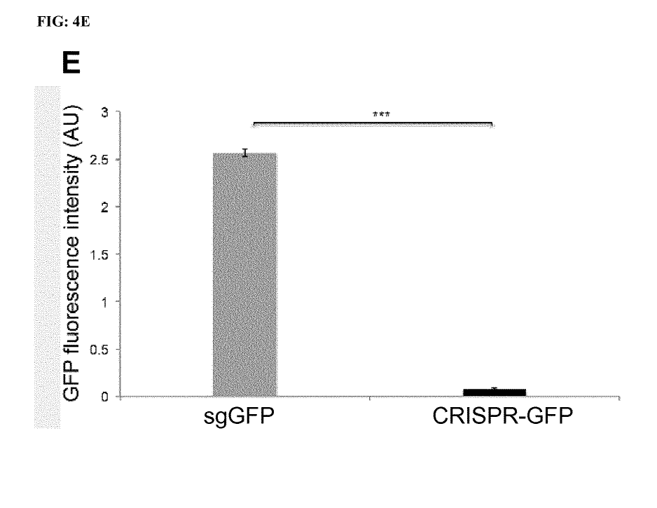

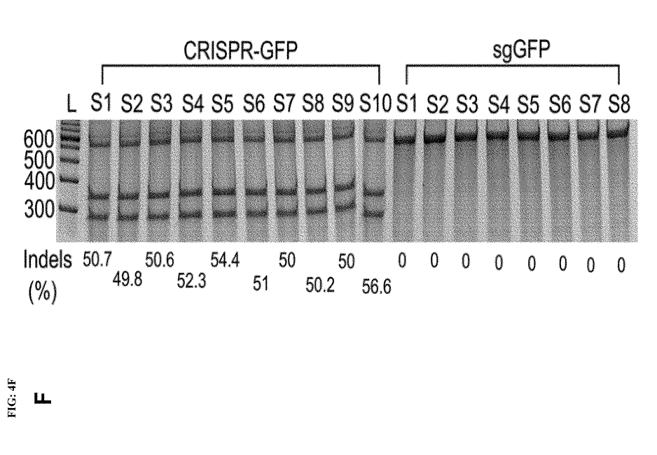

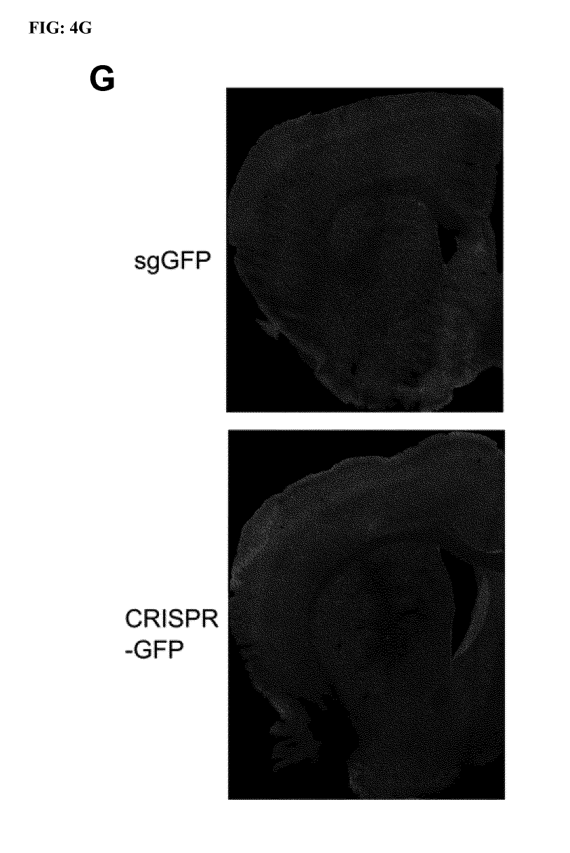

As a first step toward the establishment of an in vitro gene editing system for mHTT, Applicants performed experiments with reporter genes in HEK 293T cells. For this, a sequence containing the mCherry fluorescent protein followed by the CRISPR target sequence containing a STOP codon and the GFP gene was integrated in the genome of HEK 293T cells using LV (Examples 1-2). mCherry-positive cells were then infected with LV expressing Cas9 (SEQ ID NO: 8) and an sgRNA recognizing the target sequence close to the stop codon were designed (sgTARGET, SEQ ID NO: 2; FIG. 2A). As controls, Applicants infected cells with Cas9 or sgTARGET alone. The potency of DNA DSB appearance and NHEJ was quantified with the surveyor nuclease assay and specific restriction-length fragment polymorphisms (RFLP). Results at the DNA levels show up to 45% of DSB, 2 weeks post-infection (FIG. 3B). Using Western blots, we observed the fusion mCherry-GFP protein after NHEJ formation (FIG. 3D). Additionally, 3 days post-transfection of HEK-mCherry-GFP cells with a plasmid containing the sequence for homologous recombination without a stop codon, Applicants observed around 27% of HR (FIGS. 3C-D; SEQ ID NO: 9). The efficiency of DSB was further increased when multiple sgRNA (sgGFP1, sgGFP2 and sgGFP3, respectively SEQ ID NO: 3, 10, 11) targeting the GFP reporter gene were used (FIG. 3E). Finally, Applicants infected mouse primary cortical neuronal (FIGS. 4A-C) and astrocytic cultures (FIG. 4B; Example 3) with LV expressing Cas9, eGFP and Tomato-sgGFP1 targeting the eGFP sequence (sgGFP1). Applicants observed strong eGFP NHEJ formation at the target site couples with a loss of eGFP fluorescence in both cell populations 3 weeks post-infection (FIGS. 4A-C). This shows that Applicants' CRISPR system efficiently disrupt eGFP following DSB formation in primary cells.

Applicants evaluated the efficiency of the CRISPR system in the mouse brain to disrupt eGFP sequence (Example 5). Applicants have injected AAV (FIG. 4D) or LV (FIG. 4E-F) encoding Cas9 and sgGFP1 sequences in the striatum of mice expressing the GFP reporter gene (AAV-GFP or LV-GFP). No toxicity or adverse effects were observed up to 4 weeks post-infection in neurons, as revealed by the preservation of NeuN and DARPP-32 immunostaining (data not shown). In addition, a strong loss of GFP signal was found 3 and 4 weeks post-infection in the infected area (FIG. 4E) and gene disruption reached up to 56.6% (FIG. 4F). Importantly, eGFP-negative neurons are still expressing the GABAergic marker DARPP32, confirming that eGFP loss is due to gene disruption and not major neuronal dysfunction. Similar loss of eGFP fluorescence was observed after injection of LV-gfaABC1D(B)3-Cas9 (astrocytic tropism) and LV-Tomato-sgGFP1 in BAC GLT1-eGFP transgenic mice expressing eGFP specifically in astrocytes (FIG. 4G).

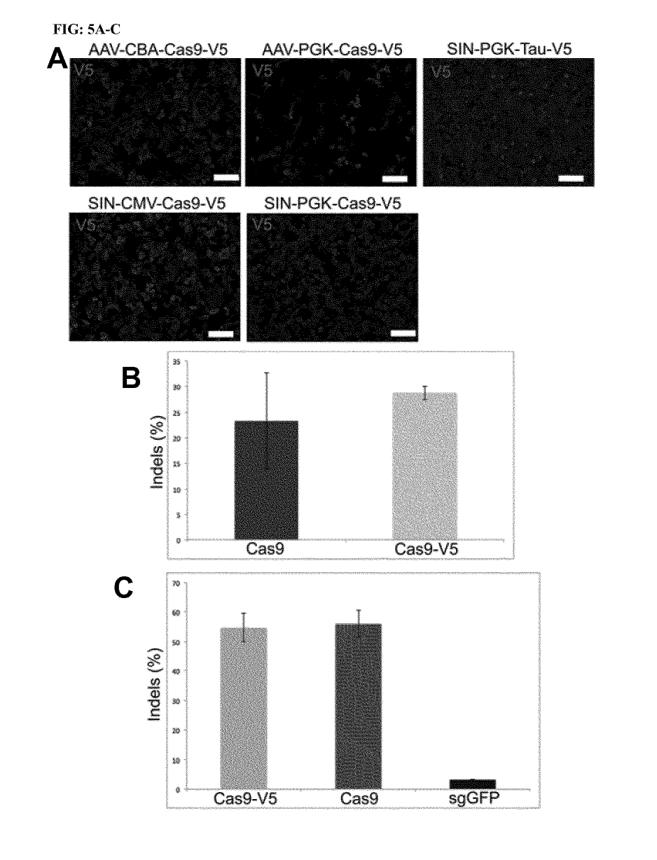

Applicants engineered a new Cas9 sequence fused to a V5-tag allowing the visualization of Cas9 protein by immunohistochemistry (Example 1, SEQ ID NO: 41). The V5-tag has been added at the 3′ end of the Cas9 gene just after the nuclear localisation signal (NLS). Expression of the Cas9-V5 tag (cloned in AAV or LV backbones) in HEK-293T cells reveal that the most of the staining is located in the cytoplasm, with some molecules are present in the nucleus (FIG. 5A). This indicates that the fusion of V5-tag close to the NLS has slightly modified the subcellular localization of Cas9, while maintaining its activity. This new property of Cas9-V5 might be particularly interesting to reach high DSB while limiting potential toxic effects in the CNS.

Applicants used gene editing with the CRISPR system as therapeutic strategy for HD. One important prerequisite for the development of in vivo gene editing/repair strategy in the CNS is an efficient delivery system. Applicants use reporter genes to develop and validate VSV-G pseudotyped LV (but other envelopes might be considered) and AAV vectors (AAV2/5, AAV2/6 and AAV-DJ, but other serotypes or chimeric AAV variants might be considered, including AAV1, 9, 10) (21). The AAV-DJ has been shown to increase spontaneous HR efficiency (22) and combined with the CRISPR system is increasing HTT editing efficiency. These gene transfer systems are suitable for in vitro and in vivo delivery (intraparenchymal or intraventricular or peripheral administrations) of the CRISPR system (data not shown). To optimize HTT gene editing, various vectors and expression cassettes were produced and tested (choice of the promoters driving the expression of the transgenes, ratio between Cas9 and sgRNA, integration of multiple sgRNA, integration of the CRISPR system in a single vector or in two separate vectors, choice of LV pseudotyping and AAV serotypes to ensure large diffusion and retrograde/anterograde transport in large areas of the CNS).

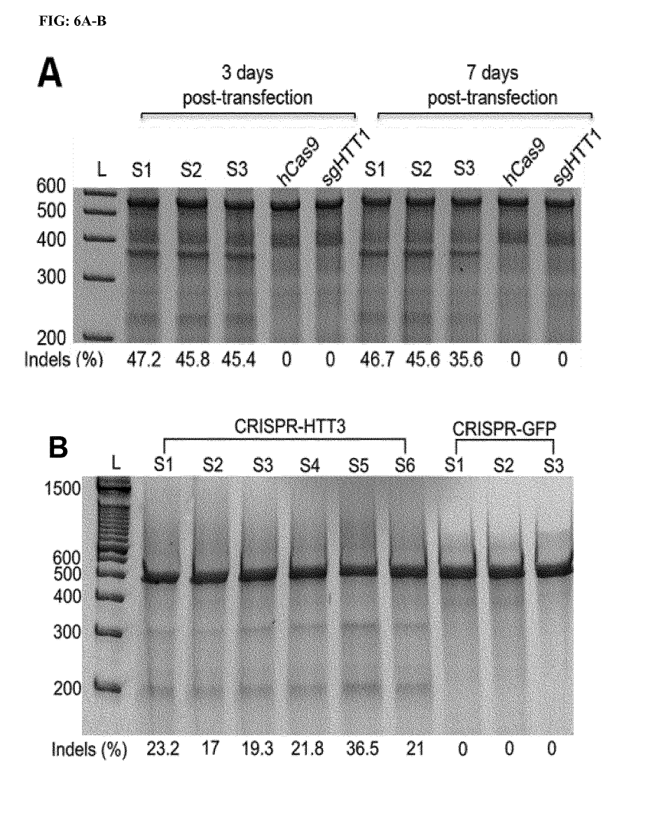

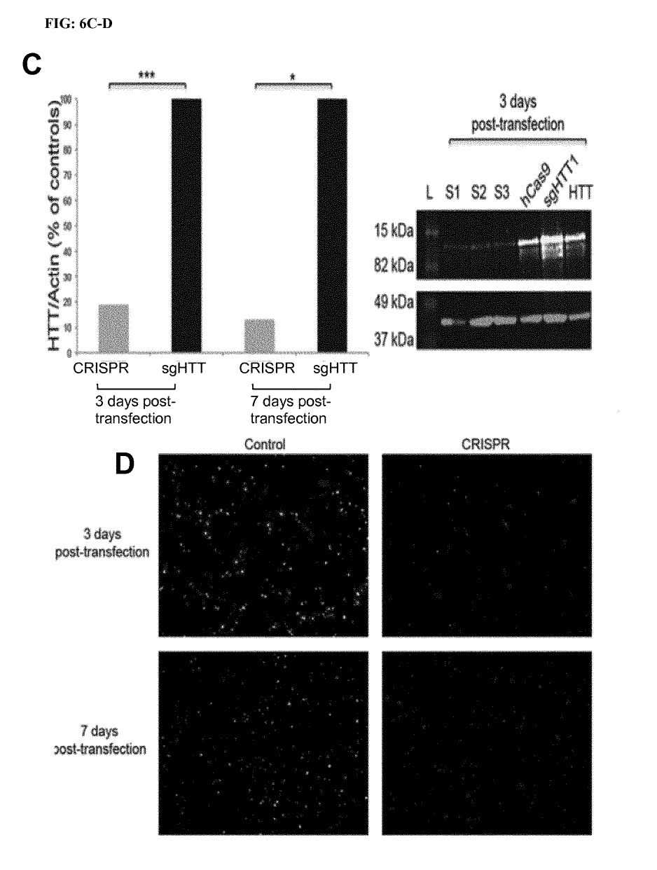

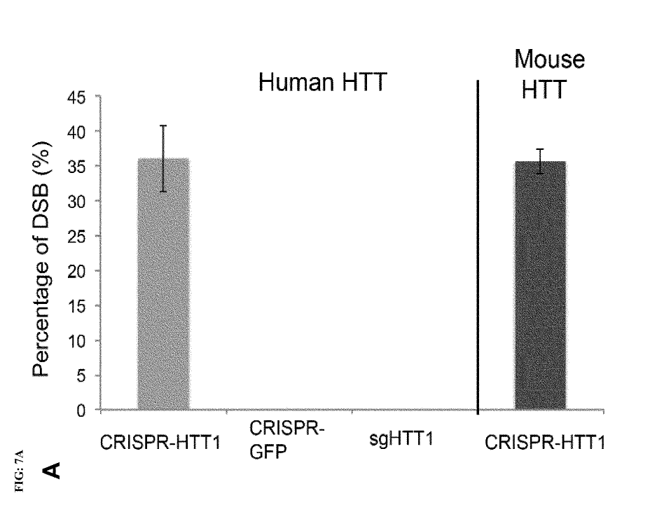

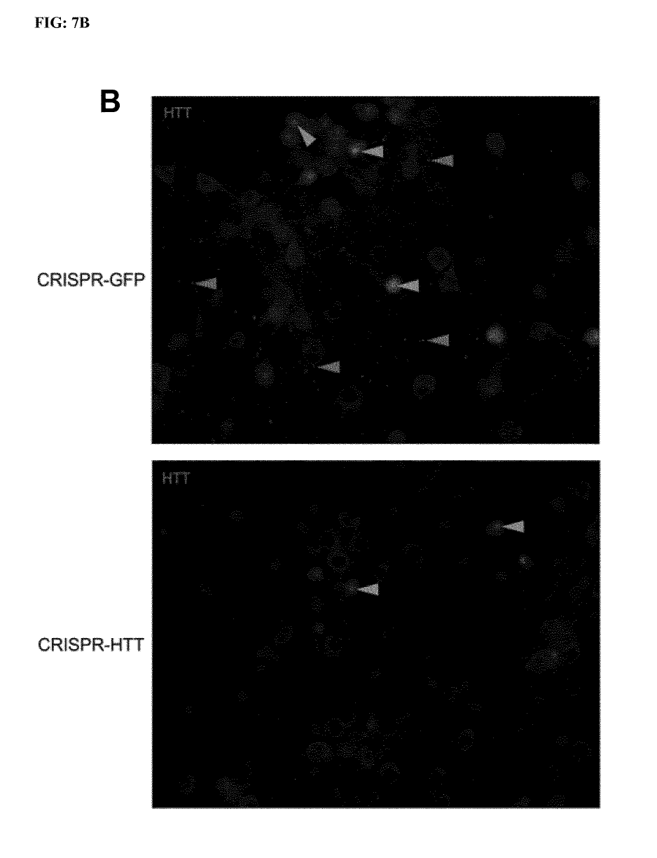

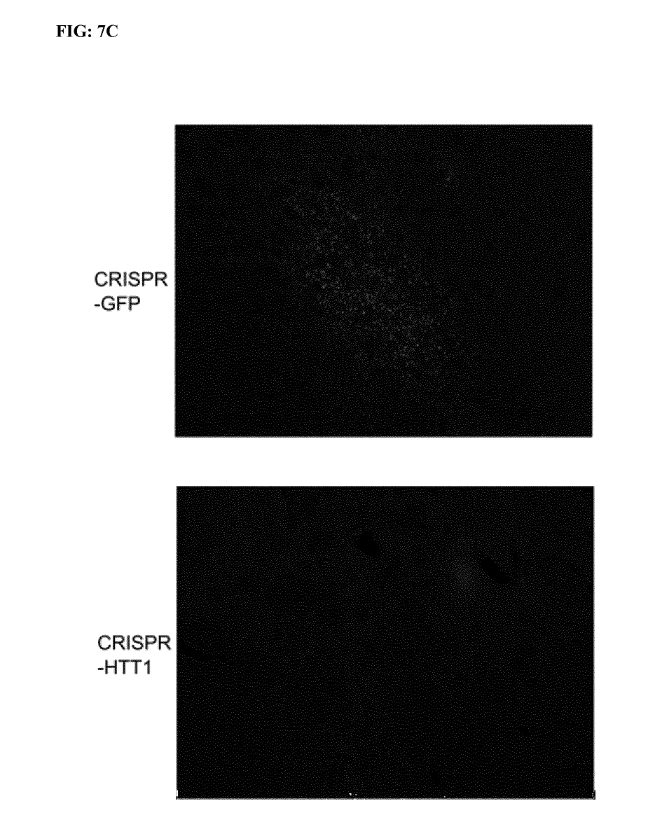

To block mutant HTT (mHTT) expression, Applicants did not use repressors (ZFN or TALEN) (WO 2013/130824) but disrupt the HTT gene (non allele- or allele-specific targeting) using DNA-targeted nucleases (CRISPR). In particular, Applicants use the recently described CRISPR system to induce mHTT double-strand break (DSB) around the transcription start site (TSS) or translation initiation codon (ATG) of the HTT gene for permanent and irreversible mHTT inactivation (Examples 3-4, FIGS. 6-7, SEQ ID NO: 4-7, SEQ ID NO: 13-39). Both DNA strand (sense or anti-sense) are targeted by the sgRNA. Species-specific or sgRNA targeting conserved region of the HTT gene in human but also rodent and non-human primates were selected (SEQ ID NO: 4-7, SEQ ID NO: 13-39). The later one will greatly facilitate the pre-clinical validation of HTT gene editing in rodent and large animal models (primates) (SEQ ID NO: 4-6, 15, 28, 37-39; FIG. 2B). To maximize NHEJ efficiency, vectors with multiple sgRNA have been produced (SEQ NO 12; FIG. 3E).

The sgHTT targeting regions around of ATG or the TSS were transfected in 293T cells (SEQ NO 4, 36). Gene editing efficiencies reaching 17-45% were observed on endogenous HTT locus (FIG. 6A-B). Transfection of human embryonic kidney cells (HEK 293T cells) with a plasmid expressing a fragment of the human mHTT (first 171 amino acids with 82 CAG repeats) fused to the green fluorescent protein (GFP) reporter gene was used to further evaluate the impact of NHEJ on the protein aggregation. In 2/3 of the cases, indels modify the reading frame of the HTT-GFP and lead to the appearance of an early STOP codon (decrease GFP expression). Surveyor, western blots and cell imaging were used to confirm that NHEJ formation with sgHTT1 leads to a decrease of HTT-GFP protein and aggregation in HEK-293T cells (Example 3-4, FIG. 6C-D).

The feasibility and efficacy of the approach was further validated in post-mitotic primary neuronal and astrocytic cultures infected with an LV expressing the first 171 amino acids of human HTT with 82 CAG and LV expressing Cas9 and the sgHTT1 (full homology for human HTT and one mismatch at the 5′ end of sgRNA with endogenous mouse HTT, FIG. 7). A strong disruption of both human and mouse HTT was observed 3 weeks post-infection of mouse cortical striatal neurons demonstrating that the CRISPR system efficiently disrupt both human and mouse HTT genes. Immunostaining of mHTT aggregates demonstrate that the treatment with the CRISPR-HTT1 vectors strongly decreased both large and small mHTT aggregate of (FIG. 7B). Similar data were observed when the CRISPR-HTT1 system was used in the mouse striatum four weeks post-injection (FIG. 7C).

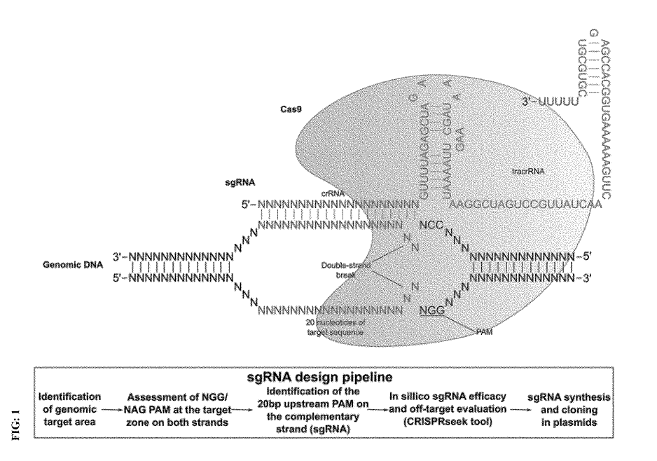

For allele-specific inactivation, Applicants selected single-nucleotide polymorphisms (SNP) located the HTT gene with high frequency of heterozygosity in the human population (>5%, preferably >15%, more preferably >20% and even more preferably >30%) (http://www.ncbi.nlm.nih.gov/projects/SNP/snp_ref.cgi?geneId=3064) with a preference for SNP located near the TSS/ATG and HD mutation and designed sgRNA targeting the disease isoform of heterozygous single-nucleotide polymorphisms (SNP; FIG. 1, 2B; SEQ ID 7, 13-35).

As a second strategy for the allele-specific gene editing of mutant HTT gene, Applicants use two sgRNAs, one targeting a SNP located close to the TSS/ATG and HD mutation of the HTT gene and a second sgRNA targeting the disease isoform of heterozygous single-nucleotide polymorphisms (SNP) located after the CAG expansion and preferentially in an intron (FIG. 2B). This leads to the complete deletion of exon 1 containing the TSS/ATG codon and expanded CAG mutation of the HTT.

For non-allele-specific or allele-specific DNA repair strategy, an sgRNA targeting any sequence located after the CAG expansion, preferentially in intron 1 of the HTT gene has been used to induce DSB and a donor sequence containing the WT human HTT exon1 and flanking sequences was used to induce homologous recombination (SEQ ID NO: 42-43). To avoid potential expression of a short HTT fragment in the recombination vector (HR donor template), the HR sequence is preferably excluding the TSS/ATG sites. For the allele-specific repair of mHTT, Applicants used sgRNA targeting the disease isoform of heterozygous single-nucleotide polymorphisms (SNP) located after the CAG expansion and preferentially in an intron (FIG. 2B-C).

Finally, as a first strategy to transiently express Cas9 protein, Applicants design an sgRNA targeting the Cas9 gene (Example 1, SEQ ID NO 40). Eliminating Cas9, should further improve the biosafety of the proposed approach. Weak promoters should be favored to favor genome editing at the beginning of the treatment and then progressively inactivating Cas9 protein.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this specification, each of which is incorporated herein by reference in its entirety.

The foregoing description will be more fully understood with reference to the following Examples. Such Examples, are, however, exemplary of methods of practicing the present invention and are not intended to limit the scope of the invention.

### EXAMPLES

### Example 1: Development of a Viral-Based Delivery System for the CRISPR System

Material and Methods

Plasmid Construction

The pcDNA3.3-TOPO-CMV-hCas9 plasmid contains the human codon-optimized Cas9 (hCas9, SEQ ID NO: 8) sequence fused to a nuclear localization signal expressed under the CMV promoter (Addgene, Cambridge Mass., USA). The hCas9 gene was excised from the plasmid using PmeI and NcoI (Roche Diagnostics GmbH, Mannheim, Germany) restriction sites and inserted into a pENTR-dual plasmid (Invitrogen, Life Technologies, Regensburg, Germany) digested with NcoI and EcoRV (Roche Diagnostics GmbH, Mannheim, Germany) (pENTR-dual-hCas9). Finally, a site-specific recombination was performed with the gateway system (Invitrogen, Regensburg, Germany; (5)) and the destination vector SIN-cPPT-PGK-RFA-WPRE, SIN-cPPT-CMV-RFA-WPRE, SIN-cPPT-gfaABC1D(b)3-RFA-WPRE, pAAV2ss-ITR-CBA-RFA-WPRE-bGHpolyA-ITR and pAAV2ss-ITR-PGK-RFA-WPRE-bGHpolyA-ITR (23) to produce the SIN-cPPT-PGK-hCas9-WPRE, SIN-cPPT-CMV-hCas9-WPRE, SIN-cPPT-gfaABC1D(b)3-hCas9-WPRE, pAAV2ss-ITR-CBA-hCas9-WPRE-bGHpolyA-ITR and pAAV2ss-ITR-PGK-hCas9-WPRE-bGHpolyA-ITR.

The pMA-T-hCas9frag-V5 containing the last 692 bp of hCas9 including the V5-tag (SEQ ID NO 8, 41, GeneArt, Invitrogen, Life Technologies, Regensburg, Germany) was digested with XhoI and XbaI and inserted in the pENTR-dual-hCas9 to replace the 3′ last nucleotides of hCas9 between the XhoI and XbaI sites (pENTR-dual-hCas9-V5, Roche Diagnostics GmbH, Mannheim, Germany). Then, a site-specific recombination was performed with the gateway system (Invitrogen, Regensburg, Germany; (5)) and the destination vector SIN-cPPT-PGK-RFA-WPRE, SIN-cPPT-CMV-RFA-WPRE, pAAV2ss-ITR-CBA-RFA-WPRE-bGHpolyA-ITR and pAAV2ss-ITR-PGK-RFA-WPRE-bGHpolyA-ITR (23) to produce the SIN-cPPT-PGK-hCas9-V5-WPRE, SIN-cPPT-CMV-hCas9-V5-WPRE, pAAV2ss-ITR-CBA-hCas9-V5-WPRE-bGHpolyA-ITR and pAAV2ss-ITR-PGK-hCas9-V5-WPRE-bGHpolyA-ITR.

The sgRNA are expressed under the control of the H1, U6 or 7SK polymerase III promoters. The H1-sgTARGET (targeting the mCherry-target-GFP sequence, SEQ ID NO 2; FIGS. 1 and 2A) was synthesized and cloned in pDONR221 vector (GeneArt, Invitrogen, Life Technologies, Regensburg, Germany). The sgRNA targeting the GFP (sgGFP1, SEQ ID NO: 3) was synthesized in a pMK-T vector (GeneArt, Invitrogen, Life Technologies, Regensburg, Germany) and transferred in pDONR221 (pDONR221-H1-sgGFP). Finally, sgRNA targeting the huntingtin (sgHTT1; SEQ ID NO 4) were synthesized as phosphorylated oligonucleotides (Microsynth AG, Balgachm, Switzerland) and annealed in a thermocycler (BioLabo, Chatel-Saint-Denis, Switzerland). The resulting double-stranded DNA was digested by using BglII and NdeI restriction sites (Roche Diagnostics GmbH, Mannheim, Germany) and inserted into the pDONR221-H1-sgRNA digested by BglII and NdeI (pDONR221-H1-sgHTT).

The plasmid containing the three sgGFP under independent polymerase III promoters was ordered from GeneArt (pENTR221-H1-sgGFP1-U6-sgGFP2-7SK-sgGFP3, GeneArt, Invitrogen, Life Technologies, Regensburg, Germany, SEQ ID NO 3,10-12). Restriction enzyme cutting were performed to produce pENTR221-H1-sgGFP1, pENTR221-H1-sgGFP1-U6-sgGFP2, pENTR221-H1-sgGFP1-7SK-sgGFP3, pENTR221-U6-sgGFP2, pENTR221-U6-sgGFP2-7SK-sgGFP3 and pENTR221-7SK-sgGFP3 (pENTR221-polIII). sgHTT2-sgHTTn were synthesized as phosphorylated oligonucleotides (MicrosynthAG, Balgachm, Switzerland) and annealed in a thermocycler (BioLabo, Chatel-Saint-Denis, Switzerland) or ordered directly as small double-stranded DNA blocks (gBlocks, Integrated DNA Technology, (IDT), Leuven, Belgium). Cloning of various sgHTT in pENTR221-polIII was done using the appropriate restriction enzyme depending on the promoter of choice (SEQ ID NO 4-7, 13-39).

A gateway reaction was used to insert each sgRNA in the transfer vectors for LV production (SIN-H1-gateway cassette-WPRE, SIN-cPPT-PGK-tdTomato-WPRE-LTR-TREtight-H1-gateway cassette, and SIN-cPPT-gateway cassette-PGK-mCherry-WHV) (5, 23, 24). For AAV, a unique Asp718 restriction site between the 5′ITR and CBA promoter of a pAAV2ss-ITR-CBA-mCherry-WPRE-bGHpolyA-ITR plasmid was digested and blunted to insert the sgRNA of choice from the pENTR221-polIII to generate the final pAAV2ss-ITR-polIII-sgRNA-CBA-mCherry-WPRE-bGHpolyA-ITR vector.

Plasmid coding for the mCherry-target-GFP sequence was designed, synthesized and cloned in pDONR221 vector (GeneArt, Invitrogen, Life Technologies, Regensburg, Germany) (pDONR221-mCherry-target-GFP) (FIG. 2A). A LR clonase (Invitrogen, Life Technologies, Regensburg, Germany) was used to insert the target sequence into a transfer vector for LV production (SIN-cPPT-PGK-mCherry-target-GFP-WPRE). The DNA template for HR with the mCherry-target-GFP reporter system (FIG. 2A) was synthesized (SEQ ID NO: 9, GeneArt, Invitrogen, Life Technologies, Regensburg, Germany) and cloned into the pDONR221 plasmid (pDONR221-template). Finally it was transferred into the pAAV2ss-ITR-DEST-RFA-bGHpoly-ITR plasmid by LR clonase reaction (kindly provided by Dr. A. Bemelmans, CEA/DSV/I2BM/MIRcen, Fontenay-aux-Roses, France) to produce the AAV2-ITR-template-bGHpolyA-ITR.

To evaluate NHEJ with sgRNA targeting the human HTT, a plasmid encoding the mutant HTT (first 171 amino acids of the human HTT with 82 CAG repeats; was fused to the eGFP (Clontech, Saint-Germain-en-Laye, France) containing 5 copies of myc tag epitope (EQKLISEEDL). This fusion construct was cloned in a third generation LV transfer vector (25) under a CMV promoter (pRRL-cPPT-CMV-Htt171-82Q-5×Myc-GFP-WPRE). The same construct without eGFP was used to generate the SIN-cPPT-PGK-Htt171-82Q-WPRE for mHTT editing in neurons. To identify infected cells in vivo, a LV transfer vector encoding the red monomer fluorescent protein mCherry was used (SIN-cPPT-PGK-mCherry-WPRE).

Cell Culture

HEK-293T cells (LGC, Wesel, Germany; human embryonic kidney cell line immortalized by the adenoviral E1A/E1B protein and expressing the SV40 large T antigen) were cultured in DMEM-Glutamax supplemented with 10% FBS and 1% Penicillin/Streptomycin (Gibco, Life Technologies, Zug, Switzerland). Cells were passed every three days using Trypsin for dissociation (Gibco, Life Technologies, Zug, Switzerland). For transfection experiments, 300′000 cells were plated in corresponding wells (6-wells; Vitaris, Baar, Switzerland) the day before transfection. Transfections were done using calcium-phosphate method. Up to 4 μg total of plasmids for 9.5 cm2 plate (6 well) were mixed in equal amount of H₂O and CaCl₂ and then added drop by drop in HEPES (Sigma-Aldrich, Buchs, Switzerland, ratio plasmid-H₂O—CaCl₂: HEPES=1:1). Mixture was incubated at RT for 5 minutes and then added to the corresponding cultures drop by drop. Medium was completely changed 6 hours post-transfection.

LV Production and Titration

LV were produced in HEK-293T cells with the four-plasmid system, as described previously (24). The HIV-1 vectors were pseudotyped with the VSV-G envelope and concentrated by ultracentrifugation and resuspended in phosphate-buffered saline (PBS, Gibco, Life Technologies, Zug, Switzerland) with 1% bovine serum albumin (BSA, Sigma-Aldrich, Buchs, Switzerland). The viral particle content of each batch was determined by p24 antigen enzyme-linked immunosorbent assay (p24 ELISA, RETROtek; Kampenhout, Belgium). The stocks were finally stored at −80° C. until use.

AAV Production

The AAV recombinant genome contains the transgene under the control of mammalian promoters, the WPRE, and the bovine hormone (bGH) gene polyadenylation signal, flanked by AAV2 ITRs. This expression cassette was encapsidated in an AAV-5, 6, DJ capsid as described previously (22, 26, 27). For this, the plasmids coding for the transgene, the capsid and Ad helper were transfected by calcium phosphate precipitation in HEK-293T cells. Cells and supernatant were harvested 3 days post-transfection and centrifuged at 300 g for 10 minutes at 4° C. Supernatant was supplemented with 8% PEG (Sigma-Aldrich, Buchs, Switzerland, 2.5M NaCl, Merck, Nottingham, UK) and was kept at 4° C. for at least 2 hours. Cell pellets were pooled and incubated in lysis buffer (NaCl 0.15M, Merck, Nottingham, UK, Tris-HCl 50 mM, pH 8.5, Sigma-Aldrich, Buchs, Switzerland) for 3 consecutive freeze/thaw cycles (30 minutes in dry ice/ethanol followed by 30 minutes at 37° C.). PEG-containing supernatant was centrifuged at 4′000 g for 20 minutes at 4° C. after the 2 hours incubation and supernatant was discarded. Cell lysate was added to the pellets and the mixture was incubated at 37° C. for 1 hour to ensure full homogenization of pellets. Lysate was then treated with benzonase (0.15 units, Sigma-Aldrich, Buchs, Switzerland) in filtered MgCl₂ 1M at 37° C. for 30 minutes (Sigma-Aldrich, Buchs, Switzerland). Treated lysate was clarified by centrifugation at 4′000 g for 20 minutes at 4° C. AAV were separated using iodixanol (AxonLab, Le Mont sur Lausanne, Switzerland) gradient ultracentrifugation at 59′000 rpm (70Ti rotor, Beckman-Coulter, Nyon, Switzerland) for 90 minutes at 20° C. as previously described. Phase containing AAV was harvested and loaded on an Amicon Ultra-15 PL 100 (Millipore, Zug, Switzerland) with 0.001% Pluronic F68 D-PBS (Gibco, Life Technologies, Zug, Switzerland) for iodixanol cleaning and viral particles concentration. Tubes were first centrifuged at 4′000 g at 4° C. until the totality of solution has passed through the column. Two additional wash with 0.001% Pluronic F68 D-PBS were done and AAV were finally resuspended in 250-500 μL 0.001% Pluronic F68 D-PBS. AAV were kept at −80° C. until the use. Titration was performed by qPCR as previously described (28).

Results

To validate and assess the efficacy of the CRISPR system, two sgRNAs targeting the mCherry-target-GFP (sgTARGET) and GFP (sgGFP1, 2, 3) sequences were designed (FIGS. 1 and 2A; SEQ ID NO: 3, 10-12). In addition, sgRNA targeting the human, rodent and primate HTT were produced (sgHTT1) (FIG. 2B; SEQ ID NO: 4). The majority of them are targeting the 5′ region, promoter, exon and intron 1 of the HTT gene to ensure loss of mutant HTT expression or repair of the mutation. The DNA templates for HR (FIG. 2C; SEQ ID NO: 9) were delivered with AAV (and in particular AAV-DJ) to ensure high number of episomes in the nucleus. Functionality of plasmids containing reporter genes was evaluated by transient transfection in HEK-293T cells (data not shown).

### Example 2: CRISPR Optimization for DSB and HR

Material and Methods

Cell Culture

HEK-293T cell culture was performed as described in the example 1. An LV-SIN-cPPT-mCherry-target-GFP-WPRE containing a target sequence and a GFP fluorescent protein (FIG. 2A) was used to infect HEK-293T cells and produce populations suitable to evaluate DSB formation (HEK-TARGET). HEK-TARGET cells were infected with the various amount of viral particles diluted in culture medium. Up to 600 ng p24 were used to infect 3×10⁵ HEK-293T cells in 9.5 cm2 wells (6 well). Cells were passed every 7 days after infection and medium was changed every 3 days.

Evaluation of the functionality of hCas9-V5 was done by transfection as described in example 1. Plasmids encoding hCas9-V5 in AAV (pAAV2ss-ITR-CBA-hCas9-V5-WPRE-bGHpolyA-ITR and pAAV2ss-ITR-PGK-hCas9-V5-WPRE-bGHpolyA-ITR) and LV backbones (SIN-cPPT-PGK-hCas9-V5-WPRE and SIN-cPPT-CMV-hCas9-V5-WPRE) were used. As a positive control for immunohistochemistry, SIN-cPPT-PGK-Tau-V5-WHV was used as previously described (29). Comparison of hCas9 and hCas9-V5 editing efficiency was done by transfecting SIN-cPPT-PGK-GFP-WHV and SIN-cPPT-PGK-Tomato-WHV-LTR-TREtight-H1-sgGFP1 and SIN-cPPT-CMV-hCas9-V5-WPRE (Cas9-V5 group) or SIN-cPPT-CMV-hCas9-WPRE (Cas9 group) in HEK-293T cells. Additive effect of multiple independent sgRNA was evaluated with SIN-cPPT-PGK-GFP-WHV, SIN-cPPT-CMV-hCas9-WPRE and SIN-cPPT-H1-sgGFP1-U6-sgGFP2-7SK-sgGFP3-PGK-mCherry-WHV (CRISPR-poly-sgGFP) or SIN-cPPT-PGK-Tomato-WHV-LTR-TREtight-H1-sgGFP1 (CRISPR-GFP).

FACS

HEK-TARGET cells were harvested and processed for FACS analysis 3-5 weeks post-infection. The cells were washed and resuspended in small volume of sterile PBS to ensure high cell concentration (1-10×10⁶/ml). For each sample, 50′000 events were analyzed. mCherry-positive cells were sorted with a Beckman-Coulter system and were grown on 96-well plates. Based on mCherry fluorescence, four distinct cell populations were isolated: cells with low copy number of target sequence (populations 1 and 10) and cells with high copy number of target sequence (populations 50 and 100).

qPCR

HEK-TARGET cells were passed and resuspended in PBS. Genomic DNA (gDNA) was extracted from 1.10⁶ cells with Trizol Reagent and resuspended at a concentration of 50 ng/μL in DNAse-free water. Provirus copy number was determined by SYBRgreen qPCR (KapaBiosystems, Le Mont sur Lausanne, Switzerland) and the primers HIV-1F-TGTGTGCCCGTCTGTTGTTGT and HIV-2R-GAGTCCTGCGTCGAGAGAGC. qPCR with primers targeting the endogenous human HTT were used as genomic standard (HTT-21F-ATGGACGGCCGCTCAGGTTCT, HTT-68R-GCTCAGCACCGGGGCAATGAA). Serial dilutions of gDNA from each population were performed and compared to the standard curve of HTT. Provirus number per cell was quantified based on the Lenti-X provirus quantitation kit (Clontech, Saint-Germain-en-Laye, France).

Primary Neuronal Cultures

Timed-pregnant FVB mice were killed by CO₂ inhalation and Ell embryos were collected in Petri dish containing HBSS. Cortex were dissected in corresponding medium containing H₂O, Na₂SO₄ 1M, K₂SO₄ 0.5M, MgCl₂ 1M, CaCl₂ 1M, HEPES 1M pH 7.3-7.4, Glucose 2.5M, Phenol red 0.5%, NaOH 1N pH 7.4 (Sigma-Aldrich, Buchs, Switzerland) and Ky/Mg (1 mM kynurenic acid/10 mM MgCl₂, Sigma-Aldrich, Buchs, Switzerland) and kept on ice in a 15 mL Falcon. Dissection medium was removed and structures were enzymatically dissociated in dissection medium containing 1 mg/mL L-cysteine and 0.125 mg/mL papain (Sigma-Aldrich, Buchs, Switzerland). Mixture was incubated at 37° C. for 10 minutes, solution was removed and the operation was repeated a second time. After the final incubation, all the papain/cysteine solution was removed and lysates were washed 3 times in dissection medium. Washed lysates were then incubated in dissection medium containing 10 mg/mL of trypsin inhibitor containing solution for 7 minutes 30 seconds at 37° C. During wash steps, overnight incubated wells in poly-D-lysine (0.019 μg/μL, Becton Dickinson, Allschwill, Switzerland) and laminin (0.027 μg/μL, Life Technologies, Zug, Switzerland) with or without glass coverslips were washed three times with H₂O and then incubated in Neurobasal supplemented with 2% B27, 1% penicillin/Streptamycin and 1% Glutamax (Gibco, Life Technologies, Zug, Switzerland) until cells plating. Lysates were then washed 3 times in dissection medium and 3 times in Opti-MEM-Glucose 2.5M (Gibco, Life Technologies, Zug, Switzerland) solution after final dissociation in Neurobasal supplemented with 2% B27, 1% penicillin/Streptamycin and 1% Glutamax (Gibco, Life Technologies, Zug, Switzerland) using Paster pipettes. Fully dissociated cells were pooled in the same tube and counted. The cells were platted at a density of 1.5×10⁵ cells per cm2 in multi-wells dishes in Neurobasal supplemented with 2% B27, 1% penicillin/Streptamycin and 1% Glutamax (Gibco, Life Technologies, Zug, Switzerland).

Different AAV serotypes were compared to determine the best delivery system for the DNA template in vitro. One day post-seeding of HEK-293T cells, 1×10⁷ Vg of AAV2/5, AAV2/6 and 5.5×10⁶ Vg AAV2/DJ-ITR-CBA-GFP-WPRE-ITR were incubated on cells. The cultures were fixed 2 weeks post-infection in 4% paraformaldehyde (Electron Microscopy Sciences, Hatfield) at room temperature (RT) for 10 minutes, washed 3 times in PBS and analyzed for direct GFP fluorescence.

Cortical neurons were infected with 20 ng/wells of LV-cPPT-PGK-GFP-WHV (LV-PGK-GFP) at DIV1. Cells were then infected with LV-cPPT-PGK-Tomato-WHV-LTR-TREtight-H1-sgGFP1 (LV-Tomato-sgGFP1, 15 ng) and LV-cPPT-CMV-hCas9-WHV (LV-hCas9, 15 ng, Cas9 group) or LV-cPPT-CMV-hCas9-V5-WHV (LV-hCas9-V5, 15 ng, Cas9-V5 group). Cells were kept in culture until DIV25.

Stereotaxic Surgery

Concentrated viral stocks were thawed on ice and resuspended by repeated pipetting. AAV were diluted in D-PBS 1× (Gibco, Life Technologies, Zug, Switzerland) to inject 9.5×10⁷ Vg in 2 μL.

Mice were anesthetized by intraperitoneal injection of a mixture of 100 mg/kg ketamine (Ketasol, Graeub, Bern, Switzerland) and 10 mg/kg xylazine (Rompun, Bayer Health Care, Uznach, Switzerland). Suspensions of AAV were injected into the mouse striatum with a 34-gauge blunt-tip needle linked to a Hamilton syringe by a polyethylene catheter. Stereotaxic coordinates were: anteroposterior +1 mm from bregma; mediolateral +/−1.5 mm and dorsoventral −3.5 mm from the skull, with the tooth bar set at −3.3 mm. Mice received a total volume of 3 μl of the vector preparation, administered at a rate of 0.2 μl/min. At the end of injections, needles were left in place for 5 min before being slowly removed. The skin was sutured with 6-0 Prolene suture (B-Braun Mediacal SA, Sempach, Switzerland) and mice were allowed to recover on a heating mat.

DNA, RNA and Proteins Extractions

DNA, RNA and proteins were extracted from the same sample with the Trizol reagent according to the manufacturer's protocol (Invitrogen, Life Technologies, Zug, Switzerland). Briefly, cells were washed in PBS (Gibco, Life Technologies, Zug, Switzerland) and lysed in Trizol. DNA and RNA were resuspended in RNAse-DNAse sterile water (Gibco, Life Technologies, Zug, Switzerland) whereas proteins were resuspended in 1% SDS, 10% Urea solution (Sigma-Aldrich, Buchs, Switzerland). RNA were kept at −80° C., proteins were kept at −20° C. and DNA diluted to 100 ng/μL were conserved at 4° C.

Genomic DNA from primary cultures was extracted using the QuickExtract DNA Extraction Solution 1.0 (Epicentre, Offenbach, Germany) following the manufacturer's instructions. Culture medium were removed and cells were incubated in Trypsin at 37° C. for 10 minutes. Cells were resuspended, diluted in 5004 of 1×PBS/well and centrifuged at 300 g for 5 minutes. Supernatant was removed, cells were resuspended in 504 of lysis buffer, each tube was vortexed for 15 secondes and incubated at 65° C. for 15 minutes with agitation. Each tube was then vortexed 15 secondes and lysis buffer was inactivated at 98° C. for 2 minutes. Genomic DNA concentration was quantified with Nanodrop (Thermo Scientific, Reinach, Switzerland) using lysis buffer as blank solution before conservation at −20° C.

Surveyor Analysis

DSB formation was evaluated using the surveyor mutation detection kit, which detects mutations and polymorphisms in DNA (Transgenomic Inc, Glasgow, UK) (30). Briefly, forward (TARGET-1F: ACGGCGAGTTCATCTACAAGGTGAAGCTGC, GFP-1F: TGACAAATGGAAGTAGCACG) and reverse oligonucleotides (TARGET-2R: TTGTACTCCAGCTTGTGCCCCAGGATGTTG, GFP-2R: GAGATGAGTTTTTGTTCGAAGGG) complementary to a region located around the target sequence in the genomic DNA (100 ng) are used with the Taq polymerase (Roche Diagnostics GmbH, Mannheim, Germany) to perform a PCR reaction. The concentration of the amplified fragment is evaluated by agarose gel electrophoresis using the GeneRuller 1 kb DNA ladder (Thermo Scientific, Reinach, Switzerland). Three hundred nanograms of PCR product is incubated at 95° C. in a thermocycler and subjected to the surveyor nuclease according to the manufacturer's protocol. Migration of the digested products is done on 10% TBE polyacrylamide precast gels (BioRad, Reinach, Switzerland). DNA is stained using SYBR gold (Molecular Probes, Life Technologies, Zug, Switzerland). The percentage of indels is determined by quantifying band intensities with Image J software (http://rsb.info.nih.gov/ij/, NIH, Bethesda) as described by Ran and collaborators (31).

Restriction Fragment Length Polymorphism (RFLP)

RFLP analysis exploits DNA variations and in particular restriction enzymes. The DNA sample is digested and the resulting restriction fragments are separated according to their lengths by gel electrophoresis. In the specific example described here, Applicants used 300-500 ng of PCR product and digest it with the restriction enzyme NcoI for 2 h at 37° C. (New England Biolabs, Allschwill, Switzerland). The resulting products were analyzed on a 10% TBE polyacrylamide precast gel (BioRad, Reinach, Switzerland) and stained in SYBR gold (Molecular Probes, Life Technologies, Zug, Switzerland). The Image J software was used to quantify the bands intensities and determine the percentage of RFLP (31).

Western Blot Analysis

Two days after transfection, the 293T cells were harvested and lysed Trizol. Protein concentration was determined by the Bradford method (Thermo Scientific, Reinach, Switzerland). Twenty micrograms of proteins were run on 12% SDS-polyacrylamide gels, and transferred to nitrocellulose membranes in 0.025M Tris, 0.192M glycine, and 20% ethanol (6). The membranes were washed 3×10 minutes in PBS and blocked 1 hour in 33% LI-COR blocking buffer in 1% PBS-Tween20 (blocking, LI-COR, Bad Homburg, Germany) at RT under agitation. Primary antibodies were incubated in blocking solution overnight at 4° C. under agitation. The following antibodies were used: anti-mCherry antibody (mCherry, 1/1000, SicGen, Lisboa, Portugal), goat anti-HTT antibody (HTT, 1/1000, SicGen, Lisboa, Portugal) and rabbit anti-Actin antibody (Actin, 1/1000, Abcam, Cambridge, UK). Membranes were rinsed 3×10 minutes in PBS-Tween20 and incubated with the following secondary antibodies in blocking for 1 hour at RT under agitation: donkey anti-goat IgG IRdye 800 (1/7000, LI-COR, Bad Homburg, Germany) and donkey anti-rabbit IgG IRdye 680 (1/7000, LI-COR, Bad Homburg, Germany). Membranes were rinsed 3×10 minutes in 1% PBS-Tween20 and 3×10 minutes in PBS before scanning on Odyssey system (LI-COR, Bad Homburg, Germany). Band intensities were determined using ImageJ software as described previously.

Immunohistochemistry

Three days post-transfection, HEK-293T cells were washed in 1×PBS and fixed in 4% PFA for 15 minutes at room temperature. Immunohistochemistry was performed as described in example 4. Mouse anti-V5 primary antibody (Invitrogen, Life Technologies, Zug, Switzerland) and goat anti-mouse 594 secondary antibody (Invitrogen, Life Technologies, Zug, Switzerland) were used.

Image Acquisitions

Pictures were acquired on a Zeiss Axiovision microscope using 20-40× objectives (Carl Zeiss Microscopy GmBH, Göttingen, Germany). Constant exposure time was used to compare fluorescence of control and treated groups.

Results

To produce cells with an integrated target sequence, HEK-293T Cells were infected with increasing doses of LV-cPPT-PGK-mCherry-target-GFP-WPRE (MOI 1, 10, 50 and 100) and selected by FACS. Based on mCherry fluorescence, four independent populations with increasing number of integrated provirus were selected (HEK-TARGET; FIG. 3A) and used for the optimization of the CRISPR system delivery. Equal amounts of LV-cPPT-CMV-hCas9-WPRE and LV-H1-sgTARGET-WPRE were incubated on each population to determine the efficiency of the CRISPR system. Cells were harvested at 3, 7, 14 and 21 days post-infection and DNA and proteins were extracted. Regions flanking the target sequence were PCR-amplified and subjected to the Surveyor mutation detection assay. In all cases, the percentage of DSB formation increased with time and is inversely proportional with the number of integrated target sequences (FIG. 3B). Importantly, cell populations carrying low (12 and 32) and high (52 and 91) provirus copies can be distinguished with Surveyor results 7 days post-infection (45% and 42% compared with 37% and 35%, respectively) but the difference is reduced at 14 days post-infection (48% and 46% compared with 43%, respectively), suggesting that a plateau has been reached. For HR delivery, we used co-transfection of the CRISPR system and the AAV2-ITR-DNA-template-bGHpolyA-ITR plasmids in cell populations 1 and 10. Cells were lysed 3 and 7 days post-transfection and subjected to RFLP with the NcoI restriction site integrated after HR (FIG. 2A). Data show an increased HR efficiency with time (respectively 21.7% and 18.1% for populations 1 and 10, 3 days post-transfection and 37.5% and 23.5% for populations 1 and 10 at 7 days post-transfection), which is corroborated by the expression of the fusion mCherry-GFP protein (FIG. 3C-D). The Applicants also evaluated the benefit of multiple transcriptionally independent sgRNA targeting the same gene. Co-transfection of plasmids coding for hCas9, Tomato-sgGFP1 and GFP in HEK-293T cells result in ˜50% of gene disruption (CRISPR-sgGFP group, FIG. 3E). However, the replacement of the Tomato-sgGFP1 plasmid by a plasmid encoding H1-sgGFP1-U6-sgGFP2-7SK-sgGFP3 (CRISPR-poly-sgGFP group) leads to a great increase of GFP gene editing up to 80% three days post-transfection (FIG. 3E). This demonstrates the added value of multiple sgRNA on DSB formation efficiency.

Applicants have evaluated the delivery of the DNA template using viral vectors in mouse primary neuronal cultures. Applicants used GFP fluorescence to assess the efficiency of gene delivery in those cells. Applicants compared AAV2/5, AAV2/6 and AAV2/DJ-ITR-CBA-GFP-WPRE-bGH polyA-ITR. Direct GFP fluorescence reveals that AAV2/5 serotype is less efficient than AAV2/6 and AAV2/DJ in vitro (data not shown). Only mild differences were observed between AAV2/6 and AAV2/DJ in both HEK-293T cells and primary neurons, revealing that these two serotypes are suitable for in vitro HR experiments. For in vivo HR experiments, AAV2/5-ITR-CBA-GFP-WPRE-bGHpolyA-ITR was injected in C57Bl/6 mice striatum based on previous studies (27). Direct fluorescence reveals strong GFP signal and large diffusion of the virus, confirming that this serotype is a potent gene transfer system for in vivo HR (data not shown).

Finally, Applicants fused the hCas9 sequence to a V5-tag to enable hCas9 labeling by immunohistochemistry (FIG. 5). Immunostaining three days post-transfection of various AAV and LV backbones expressing hCas9-V5 in HEK-293T cells reveal that all plasmids encoding hCas9 are expressed. In addition, the majority of the staining was observed in the cytoplasm with few staining in the nucleus, meaning that the addition of the V5-tag reduced the incorporation of hCas9 into the nucleus (FIG. 5A). As a consequence, editing efficiency of hCas9 and hCas9-V5 was evaluated in HEK-293T cells by transfection and in cortical neurons with LV infection. For both experiments, a GFP sequence was used as target and Surveyor was used to evaluate the rate of NHEJ. Results demonstrate that hCas9-V5 is as effective as hCas9 constructs (FIG. 5B-C).

### Example 3: CRISPR System Efficiency in Primary Neurons and Astrocytes

Material and Methods

Primary Neuronal Cultures

Mouse cortical neurons were cultured as described in Example 2.

Primary Astrocytic Cultures

Before dissection, overnight-incubated wells in poly-D-lysine (15 μg/mL, Becton Dickinson, Allschwill, Switzerland) with or without glass coverslips were washed three times with 1×PBS (Gibco, Life Technologies, Zug, Switzerland) and then incubated in 1×PBS until cells plating. P1 mouse cortex were killed by decapitation and dissected in DMEM High Glucose medium (Sigma-Aldrich, Buchs, Switzerland) dissolved in autoclaved nanopure water and supplemented with antibiotic/antimycotic (diluted 10×, Sigma-Aldrich, Buchs, Switzerland) and 3.7 g/L of sodium bicarbonate at pH 7.2 (Merck, Zug, Switzerland, DMEM-astro). Dissected cortexes were passed in 19G, 21G and 25G needle with syringe 3 times each to dissociate the structures. Cells were counted and platted in DMEM-astro at a density of 50′000 cells per cm².

Primary Culture Infections

Cortical neurons were infected with 20 ng/wells of LV-cPPT-PGK-GFP-WHV (LV-PGK-GFP) at DIV1. Cells were then infected with LV-cPPT-CMV-hCas9-WHV (LV-hCas9, 15 ng) and LV-cPPT-PGK-Tomato-WHV-LTR-TREtight-H1-sgGFP1 (LV-Tomato-sgGFP1, 15 ng, CRISPR-GFP group) or LV-hCas9 (15 ng) and LV-cPPT-PGK-mCherry-WHV (LV-mCherry, 15 ng, hCas9 group) or LV-Tomato-sgGFP1 (15 ng, sgGFP group). Cells were kept in culture until DIV25.

Cortical astrocytes were infected with 20 ng/wells of LV-cPPT-CMV-GFP-WHV (LV-CMV-GFP) at DIV7. Cells were then infected with LV-hCas9, (15 ng) and LV-Tomato-sgGFP1 (15 ng, CRISPR-GFP group) or LV-Tomato-sgGFP1 (15 ng, sgGFP group). Cells were kept in culture until DIV56.

DNA Extraction

Genomic DNA from HEK-293T cells was extracted with the Trizol Reagent as described in example 2. Genomic DNA from primary cultures was extracted using the QuickExtract DNA Extraction Solution 1.0 (Epicentre, Offenbach, Germany) following the manufacturer's instructions. Culture medium were removed and cells were incubated in Trypsin at 37° C. for 10 minutes. Cells were resuspended, diluted in 500 μL of 1×PBS/well and centrifuged at 300 g for 5 minutes. Supernatant was removed, cells were resuspended in 50 μL of lysis buffer, each tube was vortexed for 15 secondes and incubated at 65° C. for 15 minutes with agitation. Each tube was then vortexed 15 secondes and lysis buffer was inactivated at 98° C. for 2 minutes. Genomic DNA concentration was quantified with Nanodrop (Thermo Scientific, Reinach, Switzerland) using lysis buffer as blank solution before conservation at −20° C.

Surveyor Analysis

DSB formation was evaluated using the surveyor mutation detection kit, which detects mutations and polymorphisms in DNA (Transgenomic Inc, Glasgow, UK) (30). Briefly, forward (GFP-1F: TGACAAATGGAAGTAGCACG) and reverse oligonucleotides (GFP-2R: GAGATGAGTTTTTGTTCGAAGGG) complementary to a region located around the target sequence in the genomic DNA (100 ng) are used with the Kapa HiFi Hotstart polymerase (KapaBiosystems, Le Mont sur Lausanne, Switzerland) to perform a PCR reaction. For each PCR, a negative sample containing water was used. PCR products were purified with the Qiaquick PCR purification kit (Qiagen) and eluted in RNAse-DNAse free water. PCR product concentration was measured with Nanodrop (Thermo Scientific, Reinach, Switzerland) using RNAse-DNAse free water as blank. For each sample, the detected concentration in negative PCR reaction (water) was subtracted to correct for remaining oligonucleotides purification. The presence of a single PCR product was verified for each sample before further Surveyor processing. Three hundred nanograms of PCR product is incubated at 95° C. in a thermocycler and subjected to the surveyor nuclease according to the manufacturer's protocol. Migration of the digested products is done on 10% TBE polyacrylamide precast gels (BioRad, Reinach, Switzerland). DNA is stained using SYBR gold (Molecular Probes, Life Technologies, Zug, Switzerland). The percentage of indels is determined by quantifying band intensities with Image J software (http://rsb.info.nih.gov/ij/, NIH, Bethesda) as described by Ran and collaborators (31).

Image Acquisitions

Neuronal cultures were fixed in 4% paraformaldehyde (PFA) for 15 minutes at room temperature. Cells were washed 3 times with 1×PBS and coverslips were mounted on microscope slides. Pictures were acquired on a Zeiss Axiovision microscope using 20-40× objectives (Carl Zeiss Microscopy GmBH, Göttingen, Germany). Constant exposure time was used to compare fluorescence of control and treated groups.

Results

The efficiency of CRISPR system was evaluated both in cultured cortical neurons and astrocytes. Three weeks post-infection with the CRISPR vectors, genomic DNA was extracted and subjected to Surveyor assay to detect the presence of indels in the target sequence. Quantifications reveal around 45% and 30% of gene disruption in the GFP gene in neurons and astrocytes, respectively (FIG. 4A-B). Microscope acquisitions show that the majority of Tomato-positive neurons are also GFP-positive in the sgGFP group. However, Tomato-positive neurons in the CRISPR-GFP samples are mainly GFP-negative (FIG. 4C). Equivalent observations were made with astrocytic cultures (data not shown). Altogether, these data demonstrate that efficient gene disruption leading to the protein loss of function is occurring both in cultures neurons and astrocytes with LV-delivered CRISPR system.

### Example 4: Delivery and Efficacy of the CRISPR System In Vivo

Material and Methods

Animals

Adult females C57Bl/6 (Janvier, Le Genest Saint Isle, France) and males and females BAC GLT1-eGFP (kindly provided by Pr. J. Rothstein, Baltimore, Md., USA) of 10-weeks old transgenic mice expressing GFP specifically in astrocytes were used for in vivo experiments. The animals were housed in a temperature-controlled room (22° C.+−1° C.) and maintained on a 12 h light/night cycle. Food and water were available ad libitum. All experimental procedures were performed in strict accordance with the recommendations of the European Community directive (86/609/EEC) and Swiss legislation about the care and use of laboratory animals. Sample size was chosen to take into account statistical variability due to surgical procedure based on previous studies (5).

Stereotaxic Surgery

Stereotaxic surgery was performed according to procedure described in the example 2

Concentrated viral stocks were thawed on ice and resuspended by repeated pipetting. 100 ng of LV-PGK-GFP, 100 ng of LV-hCas9 and 100 ng of LV-Tomato-sgGFP1 (CRISPR-GFP group) were injected in the mouse striatum. As negative controls, 100 ng of LV-PGK-GFP and 100 ng of LV-Tomato-sgGFP1 (sgGFP group) were used. For AAV editing, the same experimental design was used except that LV-GFP was replaced by an AAV2/5-CBA-GFP-WHV-bGHpolyA (1.10⁷ vg/site).

For GFP editing in astrocytes, BAC GLT1-eGFP mice were injected with 100 ng of LV-cPPT-gfaABC1D(b)3-hCas9-WHV (LV-gfaABC1D(b)3-hCas9) and 100 ng of LV-Tomato-sgGFP1 (CRISPR-GFP group). Mice injected only with 100 ng of LV-Tomato-sgGFP1 were used as negative controls (sgGFP group).

Brain Processing

Three weeks post-lentiviral injection, the animals were killed by an overdose of sodium pentobarbital (NaCl, B-Braun, Sampach, Germany; Esconarkon, Streuli, Uznach, Germany) and transcardially perfused with a 4% PFA (Electron Microscopy Sciences, Hatfield). The brains were removed and post-fixed in 4% PFA for 12 h and then cryoprotected in 20% sucrose/PBS for 3 h and in 30% sucrose/PBS for 24 h. A sledge microtome with a freezing stage at −30° C. (Leica SM2010R, Biosystems Switzerland, Nunningen, Switzerland) was used to cut coronal sections between 20 μm thickness. Slices throughout the entire striatum were collected and stored in tubes as free-floating sections in anti-freeze solution (18% sucrose, 25% sodium azide, ethylene glycol and sodium phosphate 50 mM, pH=7.4). Slices were then stored at −20° C.

Mice used for Surveyor analysis were killed by an overdose of sodium pentobarbital (NaCl, B-Braun, Sampach, Germany; Esconarkon, Streuli, Uznach, Germany) and directly decapitated. Tomato-positive infected area was dissected under an inverted fluorescent microscope for direct genomic DNA extraction.

DNA Extraction

Genomic DNA was extracted using the QuickExtract DNA Extraction Solution 1.0 (Epicentre, Offenbach, Germany) following the manufacturer's instructions. Dissected striatum was disrupted in 50 μL of lysis buffer with an automated disruptor, each tube was vortexed for 15 seconds and incubated at 65° C. for 15 minutes with agitation. Each tube was then vortexed 15 seconds and lysis buffer was inactivated at 98° C. for 2 minutes. Genomic DNA concentration was quantified with Nanodrop (Thermo Scientific, Reinach, Switzerland) using lysis buffer as blank solution before conservation at −20° C.

Surveyor Analysis

Surveyor assay against the GFP target sequence were performed as described in the Example 3 with the same primers.

Immunohistochemistry

The following primary antibodies were used: rabbit polyclonal anti-DARPP-32 antibody (DARPP-32, 1/1000, Cell Signaling, Allschwill, Switzerland) and rabbit anti-NeuN antibody (Millipore, Schaffhausen, Switzerland). Free floating slices were rinsed 3×10 minutes in PBS (Laboratorium Dr Bichsel AG, Interlaken, Switzerland), followed by the saturation of non-specific sites in PBS containing 10% BSA or 5% NGS (Sigma-Aldrich, Buchs, Switzerland) and 0.1% Triton X100 (Fluka, Sigma-Aldrich, Buchs, Switzerland) for 1 h at room temperature (RT) under agitation. Primary antibodies were incubated overnight at 4° C. under agitation. Slices were rinsed 3×10 minutes in PBS and incubated with the secondary antibodies for 1 h at room temperature under agitation. The following secondary antibody was used: goat anti-rabbit IgG AlexaFluor 488 (1/1000, Invitrogen, Life Technologies, Zug, Switzerland). Slices were then rinsed 3×10 minutes in PBS, followed by mounting with Vectashield mounting medium for fluorescence with DAPI (Vector Lab Inc, Burlingame). For in vitro experiments, fixed cells were rinsed 3×1 minute in PBS after fixation and incubated in blocking solution (5% PBS-BSA, 0.2% Triton X100) for 20 minutes at RT. Primary antibody were incubated on cells for 1 hours at RT. Cells were washed 3×1 minutes in PBS and incubated in secondary antibody for 30 minutes at RT. The following secondary antibodies were used: donkey anti-goat IgG AlexaFluor 488 (1/1000, Invitrogen, Life Technologies, Zug, Switzerland) and goat anti-mouse IgG AlexaFluor 594 (1/1000, Invitrogen, Life Technologies, Zug, Switzerland). Coverslips were then washed 3×1 minute in PBS, incubated in DAPI for 5 minutes at RT, washed 3×1 minute in PBS and then mounted on a microscope slice in FluorSafe (Calbiochem, Allschwill, Switzerland) medium.

Image Acquisitions

Mosaics were acquired on a Zeiss Axiovision microscope using 20-40× objectives (Carl Zeiss Microscopy GmBH, Göttingen, Germany). Constant exposure time was used to compare fluorescence of control and treated groups. Acquisitions for direct GFP mean fluorescence intensity quantifications were performed with a Zeiss LSM 510 confocal at 63× magnification (Carl Zeiss Microscopy GmBH, Göttingen, Germany). Cell-by-cell analysis was done with the Image J software (http://rsb.info.nih.gov/ij/, NIH, Bethesda). The cytoplasm of each Tomato-positive neuron was manually contoured and GFP intensity was measured.

Results

Lentiviral vectors coding for hCas9 and the Tomato-sgGFP were injected separately in C57Bl/6 mice to evaluate toxicity of the system. LV-PGK-mCherry was co-injected to visualize the infected area and was used as an indirect marker of neuronal toxicity. NeuN and DARPP-32 immunoreactivity, respectively two markers of neuronal survival and functionality, were not altered in the mCherry positive area 3 weeks post-injection of sgGFP or hCas9, suggesting the absence of sgRNA or hCas9 toxicity in the mouse brain. GFP editing of genomic integrated target (LV-PGK-GFP experiment) or extrachromosomal episomes (AAV-GFP experiment) resulted in a lot of Tomato-positive cells that are GFP-negative after infection with the CRISPR system (FIG. 4D-E). Cell-by-cell GFP intensity was quantified for the LV-PGK-GFP experiment. Results reveal that Tomato-positive cells of the CRISPR-GFP group are around 97% less fluorescent than Tomato-positive cells of the sgGFP group (FIG. 4E). At the DNA level, Surveyor assay shows a rate of NHEJ ranging from 49.8 to 56.6% in the Tomato-positive area (FIG. 4F). Then, injection of an astrocytic LV expressing hCas9 (LV-gfaABC1D(b)3-hCas9) with LV-Tomato-sgGFP1 in BAC GLT1-eGFP mice expressing GFP only in astrocytes resulted in a strong loss of GFP signal in the infected area in the CRISPR-GFP group compared to the sgGFP (FIG. 4G). Altogether, these data demonstrate the low toxicity of the CRISPR system in the mouse brain and its strong potency for both genomic and episomal target sequence editing in neurons and glial cells.

### Example 5: Human HTT Disruption with the CRISPR System

Selection of sgRNA Targeting the HTT Gene

The sgHTT were designed around the HTT mutation (Ensembl accession number: ENSG00000197386, position of the first CAG repeat in exon 1: 3′074′877-3′074′879), and comprising the region upstream of the HTT gene (positions 3066800) up to the beginning of intron 2 (position 3087600). For the non-allele-specific gene editing approaches, three specific regions of interest were used for the selection of the sgRNA. Region upstream of the HTT gene including the promoter (Ensembl accession number: ENSG00000197386; positions 3066800 to 3074509), exon 1 of the HTT gene including the 5′UTR the transcription and translation start sites (TSS and ATG; Ensembl accession number: ENSG00000197386: positions 3074510 to 3075088), intron 1 of the HTT (Ensembl accession number: ENSG00000197386: positions 3075089 to 3086939), exon 2 (Ensembl accession number: ENSG00000197386: positions 3086940 to 3087023) and 5′ end on intron 2 (Ensembl accession number: ENSG00000197386: positions 3087024 to 3087600).

Cell Culture

HEK-293T cells culture, transfection and infection were performed as described in example 1 and 2.

DNA and Proteins Extraction

DNA and proteins extractions using the Trizol reagent were done as described in the example 2.

Surveyor Analysis

Concentration of the extracted DNA was measured using nanodrop (Thermo Scientific, Reinach, Switzerland). Hundred nanograms of genomic DNA from HEK-293T was PCR-amplified using Kapa HiFi polymerase (KapaBiosystems, Le Mont sur Lausanne, Switzerland) with oligonucleotides located around exon 1 of the human HTT gene (HTT-1F: TTGCTGTGTGAGGCAGAACCTGCGG, HTT-2R: TGCAGCGGCTCCTCAGCCAC, HTT-3F: CACTTCACACACAGCTTCGC, HTT-4R: TGCTGCTGGAAGGACTTGAG). The PCR products were purified on column with the High Pure PCR product purification Kit (Roche Diagnostics GmbH, Mannheim, Germany) and eluted in 10-20 μL elution buffer. The Surveyor analysis was performed as described in example 3.

Western Blots

Western blot analysis was performed according to procedure described in the example 2.

Mutant HTT Aggregates Imaging

HEK-293T Cell cultures (24 wells plates; LGC, Wesel, Germany) were grown and transfected on coverslips. Cells were fixed in 4% PFA for 10 minutes at RT, washed 2 times in PBS and mounted on microscope slides. Pictures were acquired on a Zeiss Axiovision microscope using a 10× objective and FITC channel (Carl Zeiss Microscopy GmBH, Göttingen, Germany). Constant exposure time was used to compare fluorescence of control and treated groups.

Results

Applicants first targeted the endogenous human HTT gene in HEK-293T cells using the CRISPR system. Applicants co-transfected plasmids coding for hCas9 and the sgHTT1 (CRISPR-HTT1, FIG. 6A) or sgHTT3 (CRISPR-HTT3, FIG. 6B) and assessed gene editing 3 (CRISPR-HTT1 and CRISPR-HTT3) and 7 days post-transfection (CRISPR-HTT1). Surveyor analysis shows between 17-45% of DSB formation at the expected target site 3-7 days post-transfection (FIG. 6A-BA). Applicants next co-transfected plasmids coding for hCas9, sgHTT1 and the fused HTT171-82Q-GFP sequence in HEK-293T cells to determine the impact of mHTT disruption on pathological aggregates. Western blots analysis reveal up to 90% of mHTT loss in treated groups when compared with control cells (FIG. 6C). Epifluorescence microscopy reveals the presence of GFP-positive nuclear and cytoplasmic aggregates in non-treated cells, which disappear in cells transfected with the CRISPR system (FIG. 6D). This demonstrates that mHTT disruption leads to the loss of pathological aggregates.

### Example 6: Mutant HTT Editing with the CRISPR System in Neurons

Primary Neuronal Cultures

Mouse cortical neurons were cultured as described in the Example 2.

Primary Culture Infections

Cortical neurons were infected with LV-cPPT-CMV-hCas9-WHV (LV-hCas9, 15 ng) and LV-cPPT-H1-sgHTT1 (LV-sgHTT1, 15 ng, CRISPR-HTT group) at DIV1. Neurons infected with LV-hCas9 (15 ng) and LV-Tomato-sgGFP1 (15 ng, CRISPR-GFP) or LV-sgHTT1 alone (15 ng, sgHTT1) were used as negative controls. All wells were then infected with 15 ng of LV-cPPT-PGK-Htt171-82Q-WHV (LV-mHTT) at DIV4. Cells were kept in culture until DIV25.

DNA Extraction

Genomic DNA extractions were performed as described in example 3

Surveyor Analysis

Surveyor assay was performed as described in example 3. Primers for PCR amplification of human HTT were: hHTT-1F: TGACAAATGGAAGTAGCACG, hHTT-2R: GAGATGAGTTTTTGTTCGAAGGG. Primers for PCR amplification of mouse HTT were: mmHTT-1F: CCTCCTCACTTCTTTTCTATCG, mmHTT-2R: AGCATTATGTCATCCACTACC.

Animals

Adult females C57Bl/6 (Janvier, Le Genest Saint Isle, France) of 10-weeks old were used for in vivo experiments. The animals were housed in a temperature-controlled room (22° C.+−1° C.) and maintained on a 12 h light/night cycle. Food and water were available ad libitum. All experimental procedures were performed in strict accordance with the recommendations of the European Community directive (86/609/EEC) and Swiss legislation about the care and use of laboratory animals. Sample size was chosen to take into account statistical variability due to surgical procedure based on previous studies (5).

Stereotaxic Surgery

Stereotaxic surgery was performed according to procedure described in the example 3. Concentrated viral stocks were thawed on ice and resuspended by repeated pipetting. 100 ng of LV-mHTT, 100 ng of LV-hCas9 and 100 ng of LV-cPPT-H1-sgHTT1-PGK-mCherry-WHV (LV-sgHTT1-mCherry, CRISPR-HTT group) were injected in the mouse striatum. As negative controls, 100 ng of LV-mHTT, 100 ng of LV-hCas9 and 100 ng of LV-Tomato-sgGFP1 (CRISPR-GFP group) were used.

Brain Processing and Culture Neurons Fixation

Three weeks post-lentiviral injection, the animals were killed by an overdose of sodium pentobarbital (NaCl, B-Braun, Sampach, Germany; Esconarkon, Streuli, Uznach, Germany) and transcardially perfused with a 4% PFA (Electron Microscopy Sciences, Hatfield). The brains were removed and post-fixed in 4% PFA for 12 h and then cryoprotected in 20% sucrose/PBS for 3 h and in 30% sucrose/PBS for 24 h. A sledge microtome with a freezing stage at −30° C. (Leica SM2010R, Biosystems Switzerland, Nunningen, Switzerland) was used to cut coronal sections between 20 μm thickness. Slices throughout the entire striatum were collected and stored in tubes as free-floating sections in anti-freeze solution (18% sucrose, 25% sodium azide, ethylene glycol and sodium phosphate 50 mM, pH=7.4). Slices were then stored at −20° C.

Neuronal cultures were fixed in 4% paraformaldehyde (PFA, Electron Microscopy Sciences, Hatfield) for 15 minutes at room temperature. Cells were washed 3 times with 1×PBS (Gibco, Life Technologies, Zug, Switzerland) and coverslips were mounted on microscope slides.

Immunohistochemistry

The following primary antibodies was used: goat polyclonal anti-HTT (1/1000, SicGen, Lisboa, Portugal). Free floating slices were rinsed 3×10 minutes in PBS (Laboratorium Dr Bichsel AG, Interlaken, Switzerland), followed by the saturation of non-specific sites in PBS containing 5% BSA (Sigma-Aldrich, Buchs, Switzerland) and 0.1% Triton X100 (Fluka, Sigma-Aldrich, Buchs, Switzerland) for 1 h at room temperature (RT) under agitation. Primary antibodies were incubated overnight at 4° C. under agitation. Slices were rinsed 3×10 minutes in PBS and incubated with the secondary antibodies for 2 h at room temperature under agitation. The following secondary antibody was used: donkey anti-goat IgG AlexaFluor 488 (1/500, Invitrogen, Life Technologies, Zug, Switzerland). Slices were then rinsed 3×10 minutes in PBS, followed by mounting with Vectashield mounting medium for fluorescence with DAPI (Vector Lab Inc, Burlingame).

Image Acquisitions

Mosaics and in vitro aggregates imaging were acquired on a Zeiss Axiovision microscope using 20-40× objectives (Carl Zeiss Microscopy GmBH, Göttingen, Germany). Constant exposure time was used to compare fluorescence of control and treated groups.

Results

Mutant HTT editing with the sgHTT1 was first evaluated in mouse cortical neurons. Three weeks post-infection with the LV-mHTT vector, genomic DNA was extracted for Surveyor assay. Quantifications reveal around 35% of human mutant HTT disruption in neurons (FIG. 7A). Because our sgHTT1 is homologous for mouse endogenous HTT except the first nucleotide (5′ of the target sequence), we also amplified the target sequence with mouse-specific primers for Surveyor assay. Results demonstrate that cleavages occur at the same efficiency as for human HTT, meaning that our sgHTT1 is efficiently conserved across human and mouse (FIG. 7A, right part). Immunostaining of HTT in CRISPR-GFP group reveals the presence of numerous large and small mHTT aggregates, mainly localized in nucleus/cytoplasm or neuritis, respectively (FIG. 7B). The vast majority of these aggregates disappear in the CRISPR-HTT group at DIV25, especially small aggregates. Additionally, co-injection of vectors expressing CRISPR-HTT and LV-mHTT in the mouse striatum leads to the same loss of mHTT aggregation in CRISPR-HTT samples compared to CRISPR-GFP ones three weeks post-infection (FIG. 7C). Altogether, these data demonstrate that our sgHTT1 is able to efficiently cleave HTT gene in primary neurons and to reverse mHTT aggregation both in vitro and in vivo.

### REFERENCES

1. Vonsattel J P & DiFiglia M (1998) Huntington disease. J Neuropath Exp Neurol 57:369-384.
2. Zuccato C, Valenza M, & Cattaneo E (2010) Molecular mechanisms and potential therapeutical targets in Huntington's disease. Physiol Rev 90(3):905-981.
3. Ross C A & Tabrizi S J (2011) Huntington's disease: from molecular pathogenesis to clinical treatment. Lancet Neurol 10(1):83-98.
4. Fiszer A & Krzyzosiak W J (2014) Oligonucleotide-based strategies to combat polyglutamine diseases. Nucleic Acids Res 42(11):6787-6810.
5. Drouet V, et al. (2009) Sustained effects of nonallele-specific Huntingtin silencing. Ann Neurol 65(3):276-285.
6. Drouet V, et al. (2014) Allele-specific silencing of mutant huntingtin in rodent brain and human stem cells. PLoS One 9(6):e99341.
7. Garriga-Canut M, et al. (2012) Synthetic zinc finger repressors reduce mutant huntingtin expression in the brain of R6/2 mice. Proc Natl Acad Sci USA 109(45):E3136-3145.
8. Cong L, et al. (2013) Multiplex genome engineering using CRISPR/Cas systems. Science 339(6121):819-823.
9. Jinek M, et al. (2013) RNA-programmed genome editing in human cells. eLife 2:e00471.
10. Sternberg S H, Redding S, Jinek M, Greene E C, & Doudna J A (2014) DNA interrogation by the CRISPR RNA-guided endonuclease Cas9. Nature 507(7490):62-67.
11. Merienne N, Le Douce J, Faivre E, Deglon N, & Bonvento G (2013) Efficient gene delivery and selective transduction of astrocytes in the mammalian brain using viral vectors. Front Cell Neurosci 7:106.
12. Simonato M, et al. (2013) Progress in gene therapy for neurological disorders. Nat Rev Neurol 9(5):277-291.
13. Cartier N, et al. (2009) Hematopoietic stem cell gene therapy with a lentiviral vector in X-linked adrenoleukodystrophy. Science 326(5954):818-823.
14. Palfi S, et al. (2014) Long-term safety and tolerability of ProSavin, a lentiviral vector-based gene therapy for Parkinson's disease: a dose escalation, open-label, phase 1/2 trial. Lancet 383(9923):1138-1146.
15. Weinberg M S, Samulski R J, & McCown T J (2013) Adeno-associated virus (AAV) gene therapy for neurological disease. Neuropharmacology 69:82-88.
16. Naldini L, et al. (1996) In vivo gene delivery and stable transduction of nondividing cells by a lentiviral vector. Science 272(5259):263-267.
17. Mali P, et al. (2013) RNA-guided human genome engineering via Cas9. Science 339(6121):823-826.
18. Jinek M, et al. (2012) A programmable dual-RNA-guided DNA endonuclease in adaptive bacterial immunity. Science 337(6096):816-821.
19. Hirano M, et al. (2013) Highly efficient retrograde gene transfer into motor neurons by a lentiviral vector pseudotyped with fusion glycoprotein. PLoS One 8(9):e75896.
20. Castle M J, Gershenson Z T, Giles A R, Holzbaur E L, & Wolfe J H (2014) Adeno-Associated Virus Serotypes 1, 8, and 9 Share Conserved Mechanisms for Anterograde and Retrograde Axonal Transport. Hum Gene Ther 25(8):705-720.
21. Grimm D, et al. (2008) In vitro and in vivo gene therapy vector evolution via multispecies interbreeding and retargeting of adeno-associated viruses. J Virol 82(12):5887-5911.
22. Melo S P, et al. (2014) Somatic Correction of Junctional Epidermolysis Bullosa by a Highly Recombinogenic AAV Variant. Mol Ther 22(4):725-733.
23. Deglon N, et al. (2000) Self-inactivating lentiviral vectors with enhanced transgene expression as potential gene transfer system in Parkinson's disease. Hum Gene Ther 11(1):179-190.
24. Hottinger A F, Azzouz M, Deglon N, Aebischer P, & Zurn A D (2000) Complete and long-term rescue of lesioned adult motoneurons by lentiviral-mediated expression of glial cell line-derived neurotrophic factor in the facial nucleus. J Neurosci 20(15):5587-5593.
25. Dull T, et al. (1998) A third-generation lentivirus vector with a conditional packaging system. J Virol 72(11):8463-8471.
26. Low K, Aebischer P, & Schneider B L (2013) Direct and retrograde transduction of nigral neurons with AAV6, 8, and 9 and intraneuronal persistence of viral particles. Hum Gene Ther 24(6):613-629.
27. Drinkut A, Tereshchenko Y, Schulz J B, Bahr M, & Kugler S (2012) Efficient gene therapy for Parkinson's disease using astrocytes as hosts for localized neurotrophic factor delivery. Mol Ther 20(3):534-543.
28. Aurnhammer C, et al. (2012) Universal real-time PCR for the detection and quantification of adeno-associated virus serotype 2-derived inverted terminal repeat sequences. Hum Gene Ther Methods 23(1):18-28.
29. Dujardin S, et al. (2014) Ectosomes: a new mechanism for non-exosomal secretion of tau protein. PLoS One 9(6):e100760.
30. Qiu P, et al. (2004) Mutation detection using Surveyor nuclease. Biotechniques 36(4):702-707.
31. Ran F A, et al. (2013) Genome engineering using the CRISPR-Cas9 system. Nat Protoc 8(11):2281-2308.

1. A kit for the treatment of Huntington's disease (HD) for allele or non-allele-specific huntingtin (HTT) gene editing or repair comprising:
a gene delivery vector consisting of at least one viral vector selected among adeno-associated vector serotypes (AAV) and/or lentiviral vectors (LV);
a Cas9 being human codon-optimized or fused to an epitope tag selected among the group of FLAG, His, myc, Tap, HA, V5;
at least one artificial single guide RNA (sgRNA) having a total size from 63-115 nucleotides comprising a tracrRNA sequence of 48-85 nucleotides long and a crRNA sequence of 15-30 nucleotides recognizing the sequence of the HTT gene around the expanded CAG repeat mutation (ENSG00000197386, position of the first CAG repeat in exon 1: 3′074′877-3′074′879), and comprising the region upstream of the HTT gene (position 3066800) up to the beginning of intron 2 (position 3087600), wherein said crRNA sequence binds directly upstream of the required 5′-NGG/NAG-3′ protospacer adjacent motif (PAM) and whereas said crRNA sequence base-pairs with the target HTT sequence and Cas9 mediates a double-stranded break (DSB) 3-4 bp upstream of said PAM.

2. The kit for the treatment of Huntington's disease of claim 1, wherein said gene delivery vector contains various expression cassettes comprising promoters and/or miRNA-regulated system so as to modulate transgene expression.

3. The kit for the treatment of Huntington's disease according to claim 1, wherein said AAV is a mutant vector.

4. The kit for the treatment of Huntington's disease according to claim 3, wherein said AAV is the AAV-DJ.

5. The kit for the treatment of Huntington's disease according to claim 1, wherein said Cas9 is mutated to improve the efficiency and safety.

6. The kit for the treatment of Huntington's disease according to claim 5, wherein said mutated Cas9 is a Cas9 nickase.

7. The kit for the treatment of Huntington's disease according to claim 5, wherein said mutated Cas9 is a Cas9-V5.

8. The kit for the treatment of Huntington's disease according to claim 1, wherein said gene delivery vector consists in a single vector or several vectors.

9. The kit for the treatment of Huntington's disease according to claim 1, wherein said kit comprises multiple transcriptionally independent sgRNA targeting the HTT gene.

10. The kit for the treatment of Huntington's disease according to claim 1, wherein said artificial sgRNA comprising a crRNA sequence of 15-30 nucleotides recognizing the HTT gene is human-specific or a conserved sequence between human, rodent and primates with a maximum tolerated number of mismatches between sgRNA and HTT gene of 3 in the first 8 nucleotides of the sgRNA.

11. The kit for the treatment of Huntington's disease according to claim 1, wherein said artificial crRNA sequence of 15-30 nucleotides is encoded by the sequences selected among the group of SEQ ID NO: 4-7 and 13-39.

12. The kit for the treatment of Huntington's disease according to claim 1, further comprising an exogenous DNA template having at least 95% of homology with the homology arms located 10-20 bp away from the double strand break site and with a minimum length of 100 bp and up to 2.5 kb on both side of the double-stranded break (DSB).

13. The kit for the treatment of Huntington's disease according to claim 12, wherein said exogenous DNA template for HR contains or not the HTT transcriptional start sites (TSS)

14. The kit for the treatment of Huntington's disease according to claim 13, wherein said exogenous DNA template for HR does not contain the TSS.

15. The kit for the treatment of Huntington's disease according to claim 12, wherein said exogenous DNA template for HR is selected among the group comprising SEQ ID NO: 42-43.

16. The kit for the treatment of Huntington's disease according to claim 1, for use in a method for non-allele-specific HTT inactivation of the human HTT wild-type and mutant alleles.

17. The kit for the treatment of Huntington's disease according to claim 1, for use in a method for non-allele-specific HTT gene repair based on HR with a DNA template containing a wild-type HTT sequence.

18. The kit for the treatment of Huntington's disease according to claim 1, for use in a method for allele-specific HTT inactivation of the human mutant HTT gene wherein said at least one sgRNA is capable of recognizing SNP sequences located upstream a PAM along the HTT gene with a high frequency of heterozygosity in the human population>5% (according to dbSNP or 1000 Genome Project database).

19. The kit for the treatment of Huntington's disease according to claim 1, for use in a method for mutant HTT gene repair based on HR with a DNA template containing a wild-type HTT sequence, wherein said at least one sgRNA is capable of recognizing SNP sequences located upstream a PAM along the HTT gene with a high frequency of heterozygosity in the human population>5% (according to dbSNP or 1000 Genome Project database).

20. The kit for the treatment of Huntington's disease according to claim 1, further comprising an additional transcriptionally independent artificial sgRNA capable of self-inactivating Cas9 expression after human HTT editing, wherein said additional transcriptionally independent artificial sgRNA comprises a crRNA sequence of 15-30 nucleotides which recognizes key regions driving Cas9 expression selected among the ATG and/or the promoter driving the expression of Cas9.

21. The kit for the treatment of Huntington's disease according to claim 1, for use in a method of treating neuronal and glial cells in a patient in need thereof, wherein said gene delivery vector used to deliver said Cas9 and said at least one artificial single guide RNA (sgRNA) is suitable for a local administration in the human striatum or is engineered to allow diffusion and/or transport from the striatum to the entire of the basal ganglia network of said patient.

22. The kit for use according to claim 21, wherein said gene delivery vector is capable of targeting neurons and/or glial cells.
